(19) United States
(12) Patent Application Publication
(10) Pub. No.: US 2026/0041332 A1
(43) Pub. Date: Feb. 12, 2026

(54) ON DEVICE CLOSED LOOP SCAN ASSURANCE AND UNCERTAINTY AWARE VOLUMETRIC MEASUREMENT FOR MOBILE 3D SCANNING

(71) Applicant: Seyedhesam Sadeghian-motahar, Katy, TX (US)

(72) Inventor: Seyedhesam Sadeghian-motahar, Katy, TX (US)

(21) Appl. No.: 19/360,715

(22) Filed: Oct. 16, 2025

(51) Int. Cl. A61B 5/107; A61B 5/00

(52) U.S. Cl. CPC A61B 5/1079; A61B 5/0077; A61B 5/1077; A61B 5/4872; A61B 5/7221; A61B 5/742; A61B 2576/00

(57) ABSTRACT

A system and method for quality-controlled three-dimensional volumetric measurement on mobile or head-worn devices. During acquisition, the device evaluates quantitative scan-quality metrics in real time and provides corrective guidance to address deficient regions. An on-device acceptance gate prevents export until thresholds for coverage, alignment, motion stability, and volumetric uncertainty are satisfied. Following validated capture, the system generates a watertight surface model, computes volume with quantified standard uncertainty, and records the quality context supporting acceptance. Follow-up scans are registered to a baseline so that longitudinal changes are judged against propagated uncertainty, enabling statistically reliable alerts. On devices lacking hardware depth, scale is stabilized by fusing visual-inertial mapping with anthropometric priors. The validated result, including volume, uncertainty, quality indicators, and device provenance, is digitally signed and packaged as an interoperable artifact for integration with electronic health record and remote-monitoring systems.

### FIELD OF THE INVENTION

The present disclosure relates to computer-implemented systems and methods for three-dimensional reconstruction and metrology on mobile devices. More particularly, it relates to closed-loop quality assurance during acquisition and to uncertainty-aware volumetric measurement of anatomical structures using mobile devices equipped with depth sensors, with outputs suitable for clinical decision support and remote patient monitoring.

### BACKGROUND OF THE INVENTION

Modern smartphones incorporate active depth sensing, including light detection and ranging and structured light projection, as well as passive techniques that fuse color imagery with depth inferred from motion and inertial cues. Using these sensing modalities, commodity devices can reconstruct watertight surface models of limbs and other anatomical regions at practical speeds. Published evaluations in controlled settings report good agreement with reference methods for volume estimation. Those same studies also document variability linked to user technique, coverage gaps, motion, range violations, and anatomy specific challenges such as distal extremities and curved proximal segments. In unsupervised home environments these sources of variability tend to increase, which undermines clinical repeatability for longitudinal use.

Existing mobile scanning applications typically permit a user to capture a mesh and then export it for downstream measurement. Quality is often summarized as a single score or left implicit. Current tools seldom enforce explicit, quantitative acceptance criteria prior to export, and they do not couple acquisition with corrective guidance that adapts in real time to the data actually being observed. In addition, many solutions compute a single deterministic volume from a single reconstruction and do not quantify measurement uncertainty at the level of points or vertices, nor do they propagate that uncertainty to the reported volume. Alignment of follow up scans to a patient specific baseline is commonly performed offline and without an acceptance gate tied to drift limits. The result is temporal drift in time series data and insufficient auditability, both of which limit adoption for clinical decision making.

This gap is consequential for conditions in which small but clinically meaningful volume changes must be tracked outside the clinic. Lymphedema management relies on detecting limb swelling and on monitoring response to therapy. Lipedema assessment requires consistent characterization of adipose driven contour and segmental volume. Congestive heart failure frequently presents with fluid accumulation in the lower limbs, where timely recognition of decompensation can guide diuretic therapy. More generally, remote tracking of extracellular fluid volume can reduce hospitalizations and support home based care. For these use cases, clinical stakeholders require repeatable acquisition, objective acceptance thresholds, quantified uncertainty, and longitudinal comparability that do not depend on a trained operator.

Achieving the required level of repeatability on a smartphone in a home setting introduces intertwined constraints across sensing, computation, and human interaction. Coverage must be measured rather than assumed. At each moment the device should determine which portions of the target anatomy have been observed at an acceptable range and angle of incidence and with sufficient point density and depth confidence. Acceptance should depend on minimum overall coverage, maximum allowable contiguous hole size, and limits on data acquired at grazing angles or beyond the reliable range of the depth sensor. Known pipelines that visualize coverage for texturing do not enforce clinically motivated thresholds tied to these quantities prior to export.

Uncertainty must be quantified at the level of the reconstruction itself. Depth error varies with range, incidence angle, surface reflectivity, ambient illumination, and motion. When such error is not propagated, a single reported volume obscures the reliability of the measurement. There is a need for efficient on device probabilistic resampling that perturbs points or vertices according to a sensor aware and geometry aware covariance model, repeats volume integration a modest number of times, and then reports a mean volume together with a standard deviation. Acceptance should be conditioned on absolute and relative uncertainty bounds, for example a standard deviation below a few milliliters and below a fixed percentage of the measured volume.

Human guidance should be embedded within acquisition. Without immediate feedback, novice users frequently omit blind sectors, remain too far from the subject, or scan at angles that degrade depth accuracy. Real time augmented reality overlays can color the live mesh as a coverage heatmap and present succinct instructions that update in response to the evolving data, such as step closer a small distance, tilt the device upward, or circle the calf once more. The objective is to convert a failing session into a passing session within the same attempt, without training or supervision.

An acceptance gate should operate on the device and authorize export only when all required groups of criteria are satisfied. Coverage must pass the percentage and hole limits. Quality must pass motion, tracking stability, and range compliance checks. Uncertainty must be below limits. Operational guardrails improve safety and repeatability, including hard stops when the device operates out of range for a sustained fraction of frames, hard stops on tracking resets or excessive drift, and required dwell on missing sectors rather than brief flyovers. Each accepted scan should generate an immutable audit record, including threshold values, pass or fail outcomes, and diagnostic heatmaps, with thresholds fixed per device model and software version to support regulatory evaluation by agencies such as the United States Food and Drug Administration.

Longitudinal comparability also requires registration of follow up scans to a patient specific baseline with acceptance criteria for alignment error. Without such registration, small clinical changes are confounded by pose and coverage differences across sessions. Accordingly, there remains a need for a smartphone resident, closed loop framework that integrates quantitative coverage measurement, real time user guidance, uncertainty propagation to volume, conditional export tied to predefined thresholds, and baseline registration so that at home scanning can meet the repeatability required for lymphedema and lipedema assessment, congestive heart failure monitoring, and general fluid volume tracking.

### SUMMARY OF THE INVENTION

The invention provides a device-resident, closed-loop system for acquiring, validating, and exporting clinically usable three-dimensional reconstructions of anatomical regions with quantified uncertainty and auditable acceptance criteria. The system is designed to operate on mobile or head-worn spatial computing platforms, including smartphones, tablets, augmented reality headsets, virtual reality headsets with pass-through cameras, and mixed-reality devices. Unlike conventional applications that capture a mesh and defer quality assessment to later processing, the disclosed system measures coverage and quality in real time, guides the user with corrective cues that adapt to what the device is observing, estimates volume together with a rigorous measure of uncertainty, and permits export only when predefined thresholds are satisfied. The result is a repeatable, regulator-ready acquisition workflow that can be implemented using commercially available platforms equipped with depth sensing and an inertial measurement unit.

In one aspect, the invention comprises a scan-validation layer that runs on the device throughout the acquisition session. The layer continuously maintains a geometric record of where the device has obtained reliable data over the target anatomy, as well as where the data are missing or marginal. The record can be represented as a voxel grid surrounding the anatomy, as a surface-based map bound to a live mesh, or as a hybrid data structure. A region is marked as covered only if multiple conditions are met at the same time. These conditions include point density exceeding a minimum, depth confidence above a threshold derived from a sensor model, an angle of incidence within a range known to produce accurate depth, and range compliance such that the observed surface lies within the reliable operating distance of the sensor. The layer tracks both the overall coverage percentage and the size and distribution of uncovered or thinly covered regions so that the system can enforce global and local completeness requirements. During acquisition, incoming frames can be registered to the live model using confidence-weighted alignment so that higher-confidence regions influence the fit more than lower-confidence regions.

In a second aspect, the invention delivers real-time corrective guidance that is driven by evolving coverage and quality metrics. The platform renders an augmented reality overlay on the live camera view or on the live mesh, with colorization that communicates coverage status. For example, regions already observed to threshold are rendered in green, regions with marginal coverage in yellow, and missing regions in red. The overlay can include directional cues such as arrows, footprints, or a ghosted device outline that indicate where and how the user should move. A succinct instruction line updates at a cadence appropriate for the device and the user, for example step closer a small distance, tilt the device upward, or circle the region once more. Because the guidance is generated from what the device detects in the moment, it targets the specific failure modes of the ongoing session rather than a fixed script. The objective is to convert a failing scan into a passing scan within the same attempt, without training or supervision. On head-worn devices, the overlay can be anchored to the user's view so that guidance remains visible during natural head motion, and audio or haptic cues can supplement the visual overlay for accessibility. The quality monitors can include a repeatability metric computed from sequential scans and a composite quality score that aggregates coverage, alignment, and motion terms to drive next-best-view guidance.

In a third aspect, the invention quantifies measurement uncertainty at the level of the reconstructed surface and propagates that uncertainty to the reported volume. The system associates each point, vertex, or local surface patch with a covariance that reflects expected depth error as a function of sensor range, angle of incidence, surface reflectivity, ambient illumination, and motion or tracking stability. Two complementary uncertainty engines are disclosed and may be used individually or together. A probabilistic resampling engine performs a Monte Carlo procedure in which the surface is perturbed according to the local covariance model, a modest number of realizations are generated, volume is integrated for each, and the mean and standard deviation are reported. An analytic Bayesian engine maintains a probabilistic model of surface geometry and sensor noise, updates that model with observed measurements to obtain a posterior over surfaces, and then propagates that posterior through a tractable approximation of the volume functional to produce a closed-form estimate of the mean and variance of volume. The analytic engine can reduce the number of samples required to achieve a desired confidence and can attribute contributions of different uncertainty sources in an interpretable way. In some embodiments, the uncertainty engine uses adaptive resampling that increases the number of realizations until a target confidence-interval width for volume is satisfied, thereby stabilizing gating decisions.

The acceptance gate uses the outputs of the coverage tracker, the quality monitors, and the uncertainty engines to decide whether a scan is acceptable. The gate evaluates groups of criteria that must all be satisfied. A coverage group includes a minimum total coverage percentage, a maximum allowed contiguous hole size, and a limit on the fraction of data acquired at grazing angles or beyond the reliable range. A quality group includes thresholds for motion blur, camera and depth tracking stability, and drift between the device pose and the reconstructed surface. An uncertainty group includes absolute and relative limits on volumetric uncertainty. In some embodiments, the gate ties these limits to minimum clinically meaningful change specified by the clinical protocol, for example by requiring both an absolute uncertainty below a milliliter threshold and a relative uncertainty below a fixed percentage of the measured volume, scaled by the selected significance factor. If any group fails, export is withheld and the system provides targeted guidance to improve the failing metrics, including an uncertainty heatmap overlay that highlights where additional views would most reduce uncertainty.

The invention further provides operational guardrails that improve safety, reliability, and repeatability. If the device operates outside the reliable range for more than a small fraction of frames, the system pauses acquisition and issues a corrective instruction instead of silently recording low-quality data. If tracking resets or drift exceeds a threshold, the system halts and directs the user to re-establish a stable view. When the coverage tracker identifies a missing sector, the system can enforce a brief dwell requirement so that the user maintains a steady view of that sector rather than sweeping past it. These guardrails modify user behavior in ways that improve the quality of the final reconstruction yet remain simple to follow.

In preferred implementations, the system produces an audit bundle for each accepted scan. The audit bundle is an immutable record that contains the threshold values in effect for the device model and software version, the pass or fail outcomes for each group of criteria, and the diagnostic maps or heatmaps used by the gate, such as coverage maps and uncertainty overlays. The bundle can include anonymized logs of relevant quality signals, such as range compliance over time or the number of tracking resets encountered. Persisting such records per scan supports regulatory review and post hoc verification by clinical stakeholders. Thresholds are fixed per device model and software version so that acceptance decisions are stable and explainable across sites and over time, which is desirable for evaluation by agencies such as the United States Food and Drug Administration.

In certain embodiments, the invention situates acquisition within a longitudinal protocol. Follow-up scans can be registered to a patient-specific baseline model, and alignment is itself subject to acceptance criteria. For example, the system can compute a residual alignment error over a region of interest and decline to export longitudinal metrics unless the residual falls below a limit. Physical or virtual fiducials can be used to define segment boundaries, such as mid-calf to mid-thigh, and the invention can require that both the baseline and the follow-up include sufficient coverage of these boundaries to support consistent volume integration. By combining baseline registration with the acceptance gate, the system yields time series that are robust to small pose and coverage differences that would otherwise confound clinical interpretation. For longitudinal analysis, the system can compute the uncertainty of a change in volume using an expression that accounts for covariance between measurements, and it can adjudicate significance by comparing the observed change to that uncertainty.

The invention is implementable as software executing on mobile or head-worn platforms that provide at least one color camera, an inertial measurement unit, and a depth capability. Depth capability can be provided by Light Detection and Ranging, structured-light projection, time of flight, stereoscopic imaging, or depth from motion. All critical computations, including coverage tracking, guidance generation, uncertainty estimation by probabilistic resampling or analytic Bayesian propagation, and acceptance gating, can be performed on the device in real time. The system can export only the validated reconstruction and its audit bundle, to a local application or to a remote service, once acceptance is achieved. The invention can also be embodied as a non-transitory computer-readable medium that stores instructions which, when executed by a processor of a mobile or head-worn platform, cause the device to perform the acquisition, guidance, uncertainty estimation, and acceptance gating steps described here.

### BRIEF DESCRIPTION OF THE DRAWINGS

The present disclosure can be understood with reference to the description of the embodiments set out below, in conjunction with the appended drawings in which:

FIG. 1 illustrates a functional block diagram of a mobile device configured to perform closed-loop quality assurance and uncertainty-aware volumetry.

FIG. 2 illustrates a closed-loop acquisition state machine showing quality assurance computation, augmented reality guidance, and export lockout.

FIG. 3 illustrates a coverage meter visualization and next-best-view path planner overlays during scanning.

FIG. 4 illustrates per-vertex covariance estimation based on depth confidence, range, and incidence with multi-view fusion.

FIG. 5 illustrates a divergence-theorem volume computation schematic using signed tetrahedra with a reference point.

FIG. 6 illustrates a Monte Carlo propagation flow with adaptive stopping based on confidence-interval half-width.

FIG. 7 illustrates an uncertainty heatmap mapped onto a reconstructed limb mesh.

FIG. 8 illustrates baseline alignment and confidence-weighted deformable registration for longitudinal comparison.

FIG. 9 illustrates a regional delta-volume map with quality gating of comparisons.

FIG. 10 illustrates a compression-under-load protocol and computation of a compressibility index.

FIG. 11 illustrates markerless metric scaling using SLAM in combination with anthropometric priors.

FIG. 12 illustrates packaging of validated volumetric data with signing and formatting for electronic health record and remote patient monitoring integration.

### DEFINITIONS

As used herein, unless the context indicates otherwise, the singular includes the plural and the plural includes the singular; “or” is inclusive; “comprise,” “comprises,” and “comprising” mean include but are not limited to; numerical ranges include their endpoints and intermediate values; and “about” or “approximately” allow variation attributable to measurement or manufacturing tolerances. Examples are illustrative and non-limiting. A “patient” may also be referred to as a “subject.”

“Acceptance gate” refers to a device-resident control that withholds export unless predefined thresholds for coverage, alignment, motion, and uncertainty are satisfied.

“Alignment root mean square (alignment RMS)” refers to the square root of the mean of the squared point-to-surface residuals between observed data and the current model after registration, typically expressed in millimeters.

“Anthropometric priors” refers to soft constraints on plausible anatomical dimensions used to stabilize metric scale on platforms without hardware depth.

“Baseline model” refers to a stored, validated surface model of a patient's anatomy, acquired under defined conditions and used as a reference for longitudinal alignment and comparison.

“Closed-loop” refers to a process during acquisition in which the system computes quantitative quality metrics from incoming sensor data, provides real-time corrective guidance based on those metrics, and updates the three-dimensional model based on the user's response.

“Compressibility index” refers to the percentage reduction in volume between a baseline scan and a scan acquired under a specified compression pressure, reported together with its associated uncertainty.

“Coverage” refers to the percentage of the region-of-interest surface area that has been observed with acceptable confidence and suitable incidence, computed as observed area within the region of interest divided by total region-of-interest surface area.

“Device and software provenance” refers to identifiers and version information recorded with each accepted scan to support audit and repeatability.

“Digital twin” refers to the baseline model together with associated metadata, including device and software provenance, acceptance indicators, and uncertainty summaries, persisted for subsequent longitudinal analysis.

“Divergence-theorem summation” refers to a mass-properties computation of enclosed volume on an oriented, watertight triangle mesh by summing signed tetrahedron contributions relative to a fixed reference point.

“Dwell” refers to holding a stable view of a flagged sector for at least a configured duration to satisfy a completeness requirement.

“Electronic Health Record (EHR)” refers to a clinical information system configured to store patient data and to receive and interpret the structured, validated volumetric measurement artifact described herein.

“Export lockout” refers to a conditional mechanism that prevents exporting, saving, or transmitting a computed measurement unless and until predefined validation thresholds for scan quality and measurement uncertainty are satisfied.

“Fast Healthcare Interoperability Resources (FHIR)” refers to a healthcare interoperability standard that provides a non-limiting example of a format usable to structure the exported artifact for ingestion by clinical systems.

“Minimal clinically meaningful change (MMC)” refers to a clinic-defined change threshold below which differences are not clinically actionable, specified as an absolute value or a relative percentage.

“Motion blur index” refers to a scalar sharpness measure derived from the image stream, where higher values indicate greater blur.

“Next best view” refers to a suggested device pose or motion predicted to most efficiently increase coverage or reduce local uncertainty in the region of interest.

“Per-vertex covariance” refers to a three-by-three position covariance matrix assigned to a mesh vertex that quantifies anisotropic positional uncertainty in a common coordinate frame.

“Region of interest (ROI)” refers to the portion of a subject's surface selected for volumetric analysis, such as a limb segment between defined anatomical landmarks.

“Remote patient monitoring (RPM)” refers to a workflow in which validated measurements and their associated quality metadata are transmitted from a patient's location for remote clinical review and alerting.

“Repeatability score” refers to a quantitative measure of agreement between two back-to-back scans of the same region under similar conditions, formed from the observed difference and the predicted uncertainties of the two scans.

“Significance factor k” refers to a configurable multiplier used to adjudicate whether an observed change exceeds expected random variation, for example, in an alert criterion requiring |ΔV|>k·σΔV.

“Simultaneous localization and mapping (SLAM)” refers to a reconstruction technique that estimates camera poses and a three-dimensional scene model from image and inertial data; in some embodiments, SLAM-derived scale is fused with anthropometric priors to achieve metric scale.

“Standard uncertainty of volume (TV)” refers to the standard deviation of the distribution of plausible volume values obtained by propagating sensor-level and geometric uncertainties through the volume computation.

“Uncertainty-aware” refers to a system or method that computes a primary measurement, for example a volume, and also quantifies its statistical uncertainty by propagating uncertainties through the computation, with that uncertainty used for gating or alerting.

“Uncertainty heatmap” refers to a color overlay rendered on the reconstructed surface in which each vertex color encodes a scalar derived from its covariance.

“Validated volumetric measurement” refers to the output produced after the export lockout is released, including the estimated volume, its standard uncertainty, and the set of scan-quality metrics and thresholds satisfied to validate the measurement.

### DETAILED DESCRIPTION

The following description presents representative embodiments of systems, methods, and non-transitory computer-readable media that perform on-device, closed-loop quality assurance and uncertainty-aware volumetric measurement on mobile and head-worn spatial computing platforms. The platforms include smartphones, tablets, augmented reality headsets, virtual reality headsets with pass-through cameras, and mixed-reality devices. The embodiments are provided to teach how to make and use the invention. Individual features disclosed here may be used alone or in combination unless stated otherwise. Steps may be performed in different orders or in parallel. Hardware and software components may be combined or divided without departing from the scope described.

In representative implementations, the platform includes at least one color image sensor, an optional depth sensor, an inertial measurement unit, a graphics processor, a general-purpose processor, memory, and a display capable of augmented reality overlays. Software modules execute locally and include: an acquisition engine that synchronizes and fuses sensor streams; a reconstruction engine that maintains a live surface representation of the region of interest; a quality-assurance and validation module that computes quantitative scan-quality metrics in real time; a guidance interface that issues corrective prompts; a volumetry engine that computes volume and propagates uncertainty; an acceptance gate that withholds export until predefined criteria are satisfied; and a packaging module that emits a validated and auditable artifact.

The system operates in a feedback loop. As new frames arrive, the reconstruction and quality assurance modules update coverage, alignment, motion stability, and confidence measures. The guidance interface then proposes next motions predicted to improve failing metrics. Export remains locked until acceptance criteria are met. When criteria are met, the volumetry engine reports an enclosed volume accompanied by a standard uncertainty, and the packaging module emits a digitally signed record that includes device and software provenance as well as the quality context in which the measurement was accepted.

The acquisition engine time-synchronizes color, depth, and inertial samples and estimates platform pose over time. The reconstruction engine incrementally registers incoming frames to a working model of the anatomy and updates the model's geometry and per-point confidence.

During acquisition, incoming frames can be registered to the live model using confidence-weighted alignment so that higher-confidence regions influence the fit more than lower-confidence regions. Confidence weights are derived from depth-quality signals, incidence angle relative to the surface normal, viewing range, local texture, motion blur, and the number and diversity of views that observe the same patch. Weighting reduces the influence of unreliable samples on pose estimation and model update, which in turn stabilizes the live reconstruction in the presence of occlusions, low light, or suboptimal views. In some embodiments, the per-correspondence weight is taken inversely proportional to predicted positional variance along the surface normal, for example

$${w}_{i} ∝ 1 / ( {n}_{i}^{T} {\sum }_{i} {n}_{i} + ε ) ,$$

where nᵢ is ine local normal and Σᵢ is the position covariance.

The surface representation can be a watertight, consistently oriented triangle mesh or an equivalent structure suitable for accurate volume integration. Small holes may be patched where supported by adjacent geometry to ensure watertightness before volumetry. Orientation consistency is enforced so that outward-facing normals are aligned and the signed-volume computation is well-posed.

Coverage is measured rather than assumed. The system maintains a coverage map over the region of interest using a voxel grid, per-triangle attributes, or a hybrid structure. A surface element is marked as “observed to threshold” only when simultaneous conditions are met, including minimum point density, depth confidence above a sensor-model threshold, incidence within an acceptable range, and range within the reliable operating distance of the sensor. The coverage tracker maintains both global coverage percentage and the contiguity and size of any unobserved areas so that the acceptance gate can enforce completeness at global and local scales.

The quality assurance module computes quantitative metrics in real time. Representative metrics include: surface-coverage percentage within the region of interest, alignment error expressed as a root-mean-square residual between observations and the current model, a motion-blur index derived from image sharpness and inertial stability, and depth-confidence summaries such as a percentile of per-point confidence. The system can also compute a repeatability metric from back-to-back scans taken under similar conditions and can form an explicit composite quality score that aggregates normalized coverage, alignment, motion, and confidence terms to drive next-best-view selection. In one embodiment, the composite score is S=w_{c}f_{c}+wₐfₐ+wₘfₘ+w_{q}f_{q} with nonnegative weights that sum to one, and the repeatability ratio is R=|V_{A}−V_{B}|/σ_{ΔV} with acceptance when R≤τ_{R}. In some embodiments, these terms are aggregated into a composite quality score used to select the next best view. The composite score weights terms so that guidance prefers motions predicted to increase coverage and reduce uncertainty most efficiently.

The guidance interface renders an augmented-reality overlay on the live camera view or on the live mesh. Regions already observed to threshold are colored in a first hue, marginal regions in a second hue, and missing regions in a third hue. Directional cues such as arrows, footprints, or a ghosted outline indicate where and how the user should move. A short instruction line updates at a cadence appropriate for the platform and context, for example “step closer a small distance,” “tilt upward,” or “circle the region again.” On head-worn platforms, overlays are anchored to the user's view and can be supplemented by audio or haptic prompts for accessibility and for hands-busy use.

The guidance generator can choose the next instruction by ranking candidate motions according to their predicted improvement in failing metrics, subject to comfort and safety. When the repeatability metric is enabled, guidance can suggest a second pass to verify that consecutive results agree within tolerance before export is permitted.

The system quantifies uncertainty at the level of the reconstructed surface and propagates it to the reported volume. Each point, vertex, or local surface patch is associated with a position covariance that reflects expected depth error as a function of range, incidence angle, surface reflectivity, illumination, motion stability, and multi-view redundancy. Covariances contributed by different views are expressed in a common frame and combined in information form, which tightens uncertainty when multiple high-quality views corroborate a surface location.

Two complementary uncertainty engines can be used individually or together. A probabilistic resampling engine performs a Monte Carlo procedure in which the surface is perturbed according to local covariance, a number of realizations are generated, volume is integrated for each realization, and the mean and a dispersion measure are reported. An analytic Bayesian engine maintains a probabilistic model of surface geometry and sensor noise, updates that model with observed measurements to obtain a posterior over surfaces, and propagates that posterior through a tractable approximation of the volume functional to estimate the mean and variance of volume. The analytic engine can reduce the number of samples required to meet confidence goals and can attribute contributions of different uncertainty sources in an interpretable way.

In some embodiments, an adaptive stopping rule controls the number of Monte Carlo realizations so that the half-width of a selected confidence interval for the estimated volume meets a target precision. A suitable criterion is: stop when z_{1-α/2}s_{V}/\sqrt{K}≤ε_{V}, where s_{V} is the sample standard deviation of the trial volumes {Vₖ}, K is the number of realizations, z_{1-α/2} is the normal quantile, and ε_{V} is a user-selected half-width in milliliters. In some embodiments, the uncertainty engine uses adaptive resampling that increases the number of realizations until a target confidence-interval width for volume is satisfied, thereby stabilizing gating decisions. This adaptive stopping prevents the reported uncertainty from being dominated by Monte Carlo sampling noise and allows the platform to trade computation for precision in a controllable way.

Export is conditioned on an acceptance gate that evaluates groups of criteria that must all be satisfied. A coverage group enforces minimum total coverage, maximum contiguous hole size, and limits on the fraction of data acquired at grazing angles or beyond the reliable range. A quality group enforces thresholds on motion blur, tracking stability, and alignment residual, and can monitor loop-closure consistency to bound accumulated drift when the region is scanned in a circuit. An uncertainty group enforces absolute and relative limits on volumetric uncertainty so that the measurement is precise enough to resolve changes that matter clinically. The gate can tie these limits to minimal clinically meaningful change values configured by policy.

If any group fails, export remains locked. The platform presents targeted corrective guidance. When uncertainty is limiting, the interface can display an uncertainty heatmap that highlights where additional views would most reduce uncertainty. In some embodiments, the platform renders an uncertainty heatmap that colors each surface area by the magnitude of its local positional uncertainty, with higher uncertainty shown in warmer colors, and simultaneously presents augmented reality overlays indicating the next best view to acquire. Operational guardrails pause acquisition when the platform operates out of range for a sustained fraction of frames, halt the process on repeated tracking resets or excessive drift, and require dwell over missing sectors rather than quick fly-overs. Guardrails create a consistent and explainable basis for rejecting low-quality sessions while providing concrete steps to remedy deficiencies.

The system supports longitudinal use through storage of a subject-specific baseline model and registration of follow-up scans to that baseline. Registration can be rigid or deformable. In either case, confidence-weighted objectives down-weight low-quality regions so that alignment is driven by reliable geometry. Alignment itself can be subject to acceptance criteria, for example a residual error threshold within a defined region of interest.

After alignment, the system computes a change in volume and its uncertainty. In representative embodiments, the uncertainty of change is computed by a closed-form expression that accounts for covariance between measurements:

$${σ}_{Δ V} = \sqrt{{σ}_{{V}_{1}}^{2} + {σ}_{{V}_{2}}^{2} - 2 Cov ( {V}_{1} , {V}_{2} )} .$$

When the two validated scans are treated as independent,

$$Cov ( {V}_{1} , {V}_{2} ) = 0 and {σ}_{Δ V} = \sqrt{{σ}_{{V}_{1}}^{2} + {σ}_{{V}_{2}}^{2}} .$$

For longitudinal analysis, the system can compute the uncertainty of a change in volume using an expression that accounts for covariance between measurements, and it can adjudicate significance by comparing the observed change to that uncertainty. When measurements are treated as independent, the change uncertainty equals the square root of the sum of the two variance terms. When shared bias may be present, a covariance term is included, which increases or decreases the net uncertainty depending on the sign and magnitude of the shared component. A configurable significance factor provides the alert threshold, which ties clinical notifications to statistically supported change rather than single-trial noise.

The framework is device-agnostic within the defined class of mobile and head-worn platforms. On platforms with active depth, per-pixel confidence and amplitude signals inform the covariance model. On platforms without hardware depth, the system stabilizes metric scale by fusing visual odometry with inertial measurements and by applying anthropometric priors that constrain plausible dimensions of the anatomy being scanned. Because volume scales with the cube of linear scale, explicit handling of scale reduces large volumetric biases that would otherwise arise from small scale errors. The system can segment the region of interest into anatomically meaningful compartments and report regional volumes with associated uncertainties in addition to a global result.

Calibration procedures fit parameters of the sensor- and geometry-dependent uncertainty model using phantoms across range, incidence angle, illumination, and material properties. Validation procedures verify that predicted uncertainty matches empirical spread across repeated measurements on stable phantoms within a specified band. Acceptance thresholds are fixed per device model and software version so that decisions are stable and explainable across sites and time. Thresholds can be adapted across device families to reflect capabilities, while preserving auditability through explicit recording of which thresholds were in effect.

For each accepted scan, the system produces an immutable audit record that includes the thresholds in effect, pass or fail outcomes for each criteria group, quality metrics, and summary diagnostics such as coverage maps and uncertainty overlays. The validated measurement and its context are packaged into a digitally signed artifact. The artifact includes device model and software version identifiers and may be formatted for interoperable exchange with clinical systems. Privacy is preserved through encryption and careful separation of measurement content from audit content.

All critical computations, including coverage tracking, confidence-weighted registration, guidance generation, uncertainty estimation by Monte Carlo or analytic propagation, and acceptance gating, can be performed on the platform in real time using modern mobile processors and graphics units. Non-time-critical analytics may be deferred to a remote service when permitted by policy and network conditions, while the acceptance decision remains enforced locally to preserve auditability. Guidance overlays can be complemented by audio and haptic prompts. The user interface can expose a validation report so that reviewers understand both the result and the quality context that supported export.

The following description sets forth representative embodiments with reference to the accompanying drawings to facilitate a complete understanding. Reference is now made to the drawings, in which like numerals designate like elements throughout, and each reference numeral is used consistently. Where appropriate for clarity to a non-specialist reader, abbreviated technical terms are expanded at first occurrence and principal variables are identified in context. Unless expressly stated otherwise, the embodiments and examples are non-limiting.

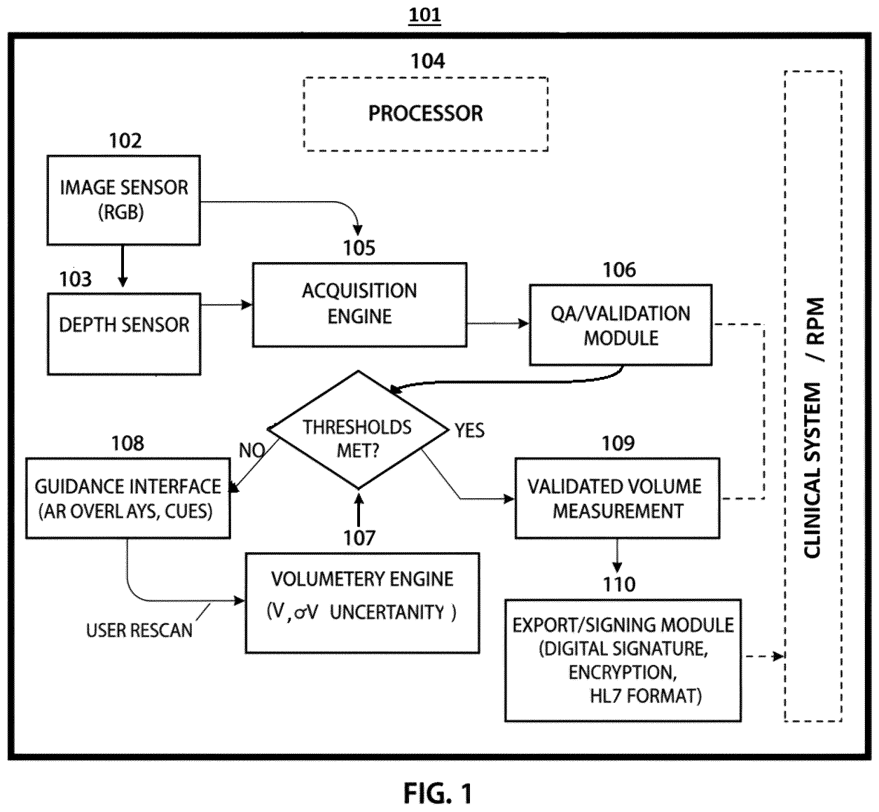

FIG. 1 depicts, in functional block form, a mobile device 101 configured to perform closed-loop quality assurance during three-dimensional capture and to compute an uncertainty-aware volumetric measurement suitable for clinical use. In the illustrated embodiment, the device 101 includes an image sensor 102, a depth sensor 103, and a processor 104 that executes an acquisition engine 105, a quality-assurance and validation module 106, a volumetry engine 107, a guidance interface 108, a validated volumetric measurement output 109, and an export and signing module 110. The foregoing components are operatively coupled such that image and depth data acquired by sensors 102 and 103 are temporally synchronized and spatially registered, evaluated for quality in real time, and processed into a validated volumetric result that is explicitly qualified by associated uncertainty prior to export. In certain embodiments, processor 104 is further configured to maintain provenance metadata for the sensed data and to cryptographically bind validation indicators to the exported result, thereby enabling downstream verification.

The image sensor 102 is configured to capture color image frames (RGB) representing visible-spectrum data of the anatomical region. The depth sensor 103 provides corresponding per-pixel range information obtained through, by way of example, light detection and ranging (LiDAR), structured illumination, stereo disparity estimation, or depth-from-motion analysis. The acquisition engine 105 time-stamps the incoming frames from sensors 102 and 103 and performs temporal synchronization such that pose-tracked color and depth samples correspond to the same physical scene instant. This synchronization mitigates motion-induced spatial misregistration that could otherwise propagate into downstream geometric reconstruction error, thereby preserving fidelity of the three-dimensional surface model and the accuracy of subsequent volumetric and uncertainty computations.

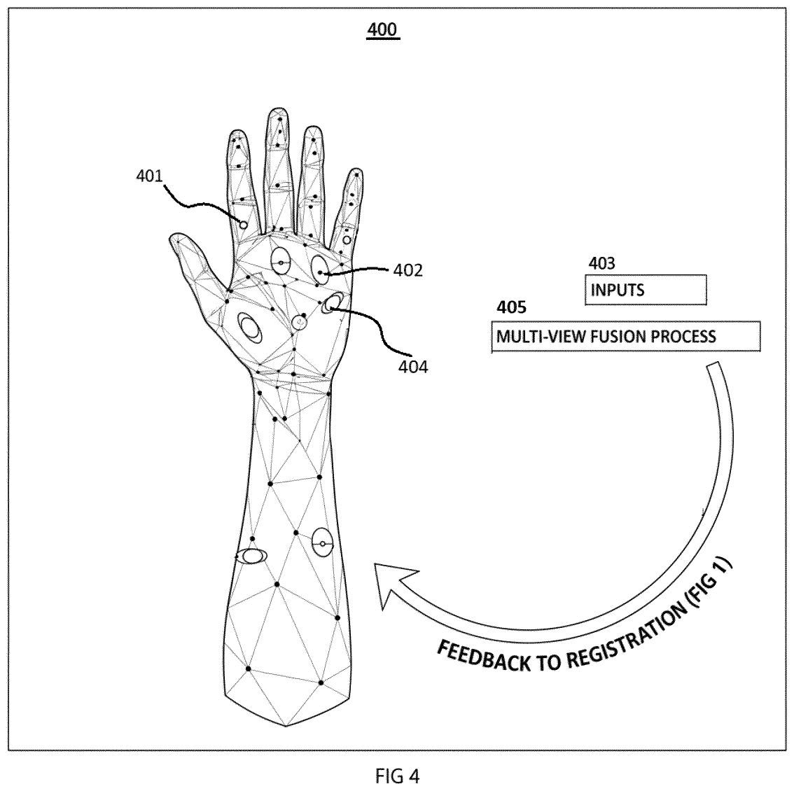

The acquisition engine 105 incrementally reconstructs a surface model of an anatomical region by aligning incoming frames to a working model. Alignment may be realized as a confidence-weighted rigid registration (e.g., a weighted Iterative Closest Point procedure) or a photometric-geometric bundle adjustment. In one embodiment executed by processor 104, the registration parameters R, t (rotation and translation) are estimated by minimizing a weighted least-squares objective:

$${min}_{R , t} {\sum }_{i} {w}_{i} {‖ {R}_{pi} + t - {q}_{i} ‖}_{2}^{2}$$

where pi are 3D points derived from the current frame, qi are corresponding points on the evolving model surface, and qi are corresponding points on the evolving model surface, and wⱼ∈[0,1] are confidence weights derived from depth quality, incidence angle, and motion-blur metrics (see FIG. 4 for covariance-based weighting). Down-weighting unreliable samples stabilizes pose estimation and prevents low-quality observations from biasing the reconstruction.

The Quality Assurance validation module 106 continuously computes quantitative scan-quality metrics from the evolving model and the incoming frames. In representative implementations, the metrics include: (i) surface-coverage percentage over a defined region of interest, (ii) alignment error expressed as root-mean-square (RMS) point-to-surface residuals, (iii) a motion-blur index, (iv) an average or percentile depth-confidence measure, and (v) a repeatability score derived from back-to-back scans where enabled. The module 106 compares each metric to predefined thresholds that may be configured per anatomy and use case. If one or more metrics are below threshold, export remains locked and the guidance interface 108 is engaged to direct corrective acquisition.

The guidance interface 108 presents augmented-reality overlays, directional cues, audio prompts, or haptics that indicate next-best views, poses predicted to increase coverage or reduce local uncertainty. In some embodiments, the intensity or frequency of prompts scales with the magnitude of the deficit relative to thresholds so that corrective feedback is proportional yet not intrusive.

The volumetry engine 107 constructs or repairs a watertight, consistently oriented triangle mesh of the region of interest and then computes (a) a base volume V from that mesh and (b) a standard uncertainty σV that quantifies expected random error for the measurement. For clarity in this specification: V denotes the scalar volume in milliliters or cubic centimeters; σV denotes the standard uncertainty (the standard deviation of plausible volume values given observed data quality). For mesh vertex j, μj∈R² denotes the best-estimate 3D position and Σj∈R^{3×3} denotes a position covariance that captures anisotropic uncertainty (units of mm²). The base volume V may be computed by summing signed tetrahedron contributions with respect to a fixed reference point (e.g., mesh centroid) so that each oriented triangle (a, b, c) contributes

$$VT = \frac{1}{6} [ ( a - o ) \times ( b - o ) ] \cdot ( c - o ) ,$$

and V=ΣVT over the mesh. The uncertainty σV may be obtained by propagating the per-vertex covariances Σj via Monte Carlo sampling in which vertices are perturbed as vj, k˜N (μj, Σj), trial volumes Vk are recomputed using the same tetrahedra summation, and σV is taken as the sample standard deviation of {Vk}. The number of trials can be adaptively increased until a confidence-interval half-width for V (or for σV) falls below a configured tolerance to ensure that the reported uncertainty is not dominated by sampling noise.

When the QA validation module 106 determines that thresholds are satisfied including, in certain clinical modes, both an absolute uncertainty gate and a relative uncertainty gate the validated volumetric measurement 109 is emitted. The export or signing module 110 assembles V, σV, the per-scan QA metrics (and indicators of which thresholds were met), and device or software provenance into a structured record, applies a digital signature, and optionally encrypts the payload for transmission or storage in external systems. The inclusion of provenance permits downstream verification that the measurement was acquired and processed under declared conditions and that acceptance criteria were met at the time of capture.

By integrating synchronized acquisition (engine 105), continuous quantitative QA with gating (module 106), user guidance to improve deficient regions (interface 108), and uncertainty-aware volumetry (engine 107) prior to export (module 110), the device 101 implements a feedback-controlled metrology workflow on a handheld platform, thereby improving repeatability and reliability relative to post-hoc, best-effort scanning.

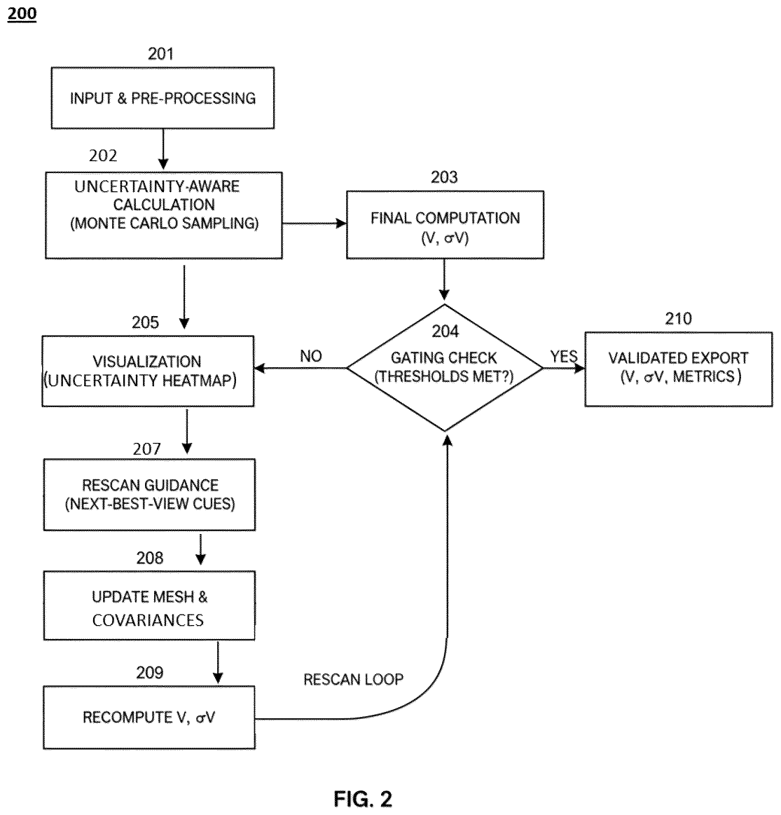

FIG. 2 illustrates, in flow-diagram form, a representative closed-loop processing pipeline 200 configured to enforce quantitative quality criteria prior to data export. The pipeline comprises an input and pre-processing stage 201, an uncertainty-aware volumetric computation stage 202, a final computation stage 203, a gating and validation check 204, a visualization and corrective rescan branch including stages 205, 207, 208, and 209, and a validated export stage 210. The sequential and conditional flow of these stages ensures that only data meeting predefined validation thresholds are permitted to advance to final export, thereby maintaining traceable measurement integrity.

At stage 201, the device acquires temporally synchronized color and depth frames, estimates camera poses through visual-inertial tracking or equivalent techniques, and incrementally updates a triangulated surface model representing the region of interest. Minor discontinuities or voids in the reconstructed surface are automatically interpolated or patched using adjacent geometric context to achieve a watertight mesh representation, and the model's vertex normals are oriented consistently to define an outward-facing surface topology suitable for volumetric computation and subsequent quality validation. For each mesh vertex j, the processor assigns a best estimate position μj∈R²³ and a three by three position covariance Σj∈R^{3×3} using depth-quality statistics and viewing geometry, as further described with reference to FIG. 4.

At stage 202, the volumetry engine propagates geometric uncertainty to the scalar volume by Monte Carlo sampling. For trial k, every vertex is perturbed according to

$${v}_{j , k} ∼ N ( μ j , \sum j ) ,$$

and the trial mesh is formed with the same connectivity and orientation. The enclosed volume for that trial, Vₖ, is recomputed, producing a set

$${\{ {V}_{k} \}}_{k = 1}^{K}$$

that characterizes how volume varies under the observed uncertainty.

At stage 203, the processor forms statistical estimators from {Vₖ}. The estimated volume is

$$V = \frac{1}{K} {\sum }_{k = 1}^{K} {V}_{k} ,$$

and the standard uncertainty is

$$σ V = \sqrt{\frac{1}{K - 1} {\sum }_{k = 1}^{K} {( {V}_{k} - V )}^{2}}$$

A two-sided confidence interval may be reported as V±z_{α} σV. For approximately ninety-five percent confidence, z_{0.975}≈1.96. To ensure that σV is not dominated by sampling noise, the device adaptively increases K until the half-width of the Monte Carlo confidence interval on V or on σV is less than or equal to a configured tolerance, for example two milliliters.

At stage 204, the system evaluates quantitative acceptance criteria that govern permission to export. The criteria include a coverage percentage greater than or equal to a predefined threshold, an alignment error expressed as a root mean square residual less than or equal to a predefined threshold, a blur index less than or equal to a predefined threshold, optionally a repeatability score within a tolerance band, and two uncertainty gates requiring σV≤X milliliters and σV/V≤Y percent. Export remains disabled unless all enumerated criteria are concurrently satisfied.

If any criterion fails at 204, the process transitions to a visualization and corrective rescan branch 205, 207, 208, 209. At 205, the device renders an uncertainty heatmap on the reconstructed mesh that maps a scalar function of each vertex covariance, for example a largest eigenvalue or a predicted variance along the surface normal, to a color scale so that high uncertainty regions are emphasized. At 207, the device presents next best view guidance that proposes camera motions predicted to increase coverage and reduce uncertainty in the emphasized regions. As additional views are acquired, the mesh and associated covariance representations are updated at 208, sampling and volume recomputation are repeated at 209, and control returns to 204. This closed-loop sequence continues until the acceptance criteria are met or the acquisition attempt is terminated.

When all criteria at 204 are satisfied, a validated export 210 is generated. The export includes, at a minimum, the estimated volume V, the standard uncertainty σV, per-scan quality metrics with indicators identifying which thresholds were met, and device and software provenance. The payload is digitally signed and, when configured, encrypted, then transmitted or stored for clinical use. In longitudinal operation, the device may compute a change in volume ΔV between validated scans together with a change uncertainty σΔV, and apply a decision rule |ΔV|>kσΔV, for example k=3, prior to flagging a clinically meaningful event.

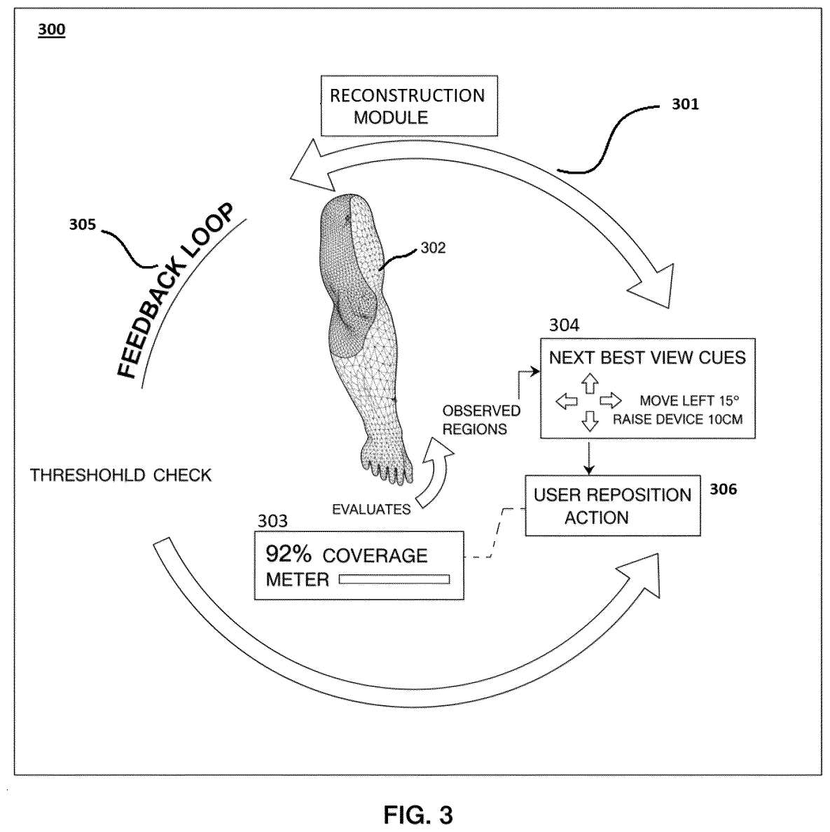

FIG. 3 is a diagram 300 illustrating a coverage visualization module 301 operating on a reconstructed mesh 302, a coverage meter 303, next best view cues 304, a feedback loop 305, and a user reposition action 306. At element 301, the device evaluates the proportion of the region of interest on mesh 302 that has been imaged with adequate confidence and suitable incidence. Triangles that satisfy those conditions are marked as observed. The device then computes a coverage percentage as

$$Coverage ( \% ) = 100 \times \frac{Area of observed triangles within ROI}{Total ROI surface areaA}$$

The coverage meter 303 presents the computed coverage value as a quantitative progress indicator accompanied by an explicit numeric percentage, for example, ninety-two percent. The acceptance threshold may be configured according to anatomical region, such as requiring at least ninety-five percent coverage for an upper limb.

At element 304, the device determines next best view directions by analyzing the spatial distribution of unobserved or low-confidence surface regions. Guidance is rendered as augmented reality overlays that include directional arrows and concise prompts, for example, “move left fifteen degrees” or “raise the device ten centimeters,” thereby enabling the user to follow explicit positional instructions without interpreting technical metrics.

At element 305, the feedback loop represents the dynamic interaction in which guidance prompts, derived from the current quality state, elicit a user reposition action 306. This action modifies the device's vantage point, expands the observed surface area, and reduces local uncertainty. The closed-loop process repeats until the coverage meter 303 attains the configured threshold, at which point the system attenuates further guidance to minimize cognitive load and transitions the scan to subsequent validation and export stages.

In practical operation, next best view guidance has proven particularly effective for anatomical regions frequently omitted in volumetric limb scans, such as the posterior calf or axillary area. By explicitly directing the user toward these incompletely captured regions, the device mitigates the risk of producing models that appear complete yet contain unobserved surfaces.

FIG. 4 is a diagram 400 depicting a reconstructed mesh 401 composed of vertices 402, inputs 403 influencing uncertainty, per-vertex covariance ellipsoids 404, and a multi-view fusion process 405. At element 401, the mesh is represented as a collection of vertices 402 connected by polygonal faces. For each vertex 402, the device establishes a local coordinate frame aligned with the viewing ray and corresponding surface tangents. Within this local frame, the device assigns a diagonal covariance matrix that quantifies anisotropic positional uncertainty.

$$diag ( {σ}_{‖}^{2} , {σ}_{⊥ 1}^{2} , {σ}_{⊥ 2}^{2} ) ,$$

where

$${σ}_{‖}^{2}$$

is the variance along the viewing direction and

$${σ}_{⊥ 1}^{2} , {σ}_{⊥ 2}^{2}$$

are variances along two orthogonal tangential directions. These values are estimated from the inputs 403, which include depth-sensor confidence at the corresponding pixel, range r from the sensor to the surface, incidence angle θ between the surface normal and the viewing ray, a motion-blur measure derived from the image stream, and the number and distribution of distinct views that observe the same surface patch. The ellipsoid 404 visually encodes this uncertainty, with elongation along directions having larger variance.

The device rotates each local covariance into a common world coordinate frame so that covariances from different views can be directly compared and fused. For a vertex j observed in a single view, the world-frame covariance is given by

$${\sum }_{j}^{( view )} = {R}_{j} diag ( {σ}_{‖}^{2} , {σ}_{⊥ 1}^{2} , {σ}_{⊥ 2}^{2} ) {R}_{j}^{T} ,$$

where Rⱼ aligns the local axes at vertex j to the world axes and

$${\sum }_{j}^{( view )}$$

is the convenience of vertex j expressed in the world frame for a single view.

When a surface patch is observed from multiple views indexed by v=1 to N, and the associated measurement noises are modeled as independent, the per-view covariances are combined in information form so that information is additive. The fused covariance satisfies

$${( {\sum }_{j}^{( fused )} )}^{- 1} = {\sum }_{v = 1}^{N} {( {\sum }_{j , v}^{( view )} )}^{- 1}$$

This expression states that each high-quality view tightens the fused uncertainty, while a low-quality view contributes little because its information matrix is small. The fused covariance

$${\sum }_{j}^{( fused )}$$

is converted to a scalar confidence for use in registration and quality scoring. In one implementation, the device maps either the largest eigenvalue

$$λmax ( {\sum }_{j}^{( fused )} )$$

or the predicted variance along the surface normal

$${n}_{j}^{T} {\sum }_{j}^{( fused )} {n}_{j}$$

to a weight wᵢ between zero and one, with larger variance producing a smaller weight and smaller variance producing a larger weight. These weights are supplied to the registration objective described with reference to FIG. 1, so that surface regions exhibiting higher reliability exert greater influence on the alignment than regions exhibiting higher uncertainty. For clarity, covariance characterizes statistical spread. If the same scan were repeated under similar conditions, a vertex associated with a larger ellipsoid 404 would yield a wider cloud of plausible positions, which indicates lower positional certainty for that vertex.

Element 405, the multi-view fusion process, combines the per-view covariances for a given vertex into a single, tightened uncertainty by accumulating information from each independent view. The device first expresses every per-view covariance in a common world coordinate frame, then aggregates the covariances in information form with numerical damping to maintain conditioning, and optionally rejects outlier views using a Mahalanobis-style consistency test. The fused result reflects increased confidence where multiple high-quality views overlap and limited change where views are weak or inconsistent. A scalar confidence value is then derived from the fused covariance and mapped to a registration and quality-scoring weight, so that reliable regions influence pose estimation and acceptance metrics more strongly than uncertain regions.

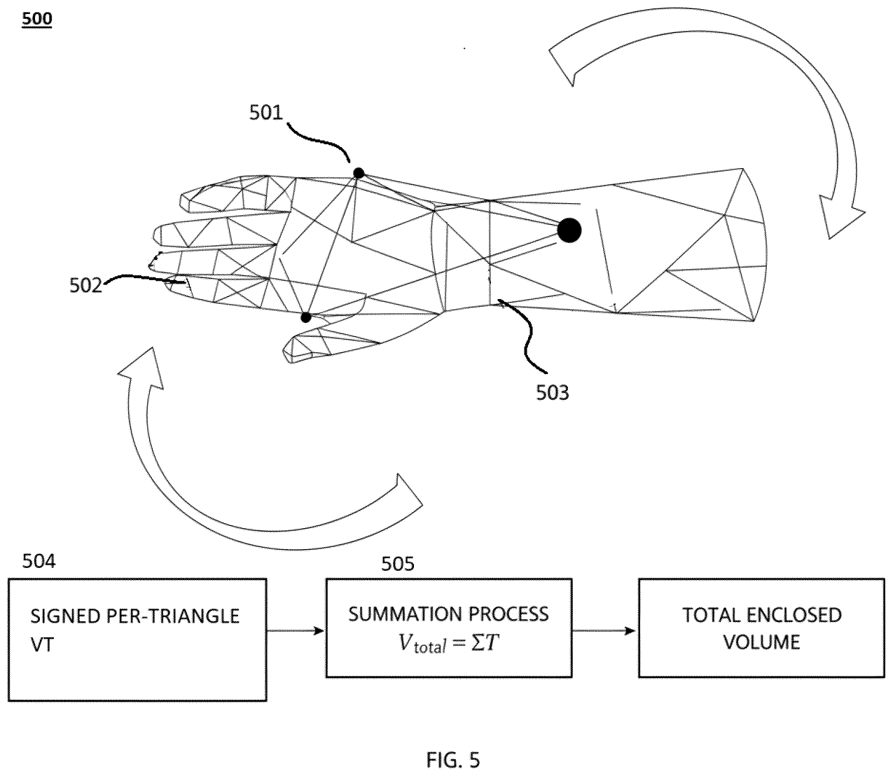

FIG. 5 is a diagram 500 depicting a watertight mesh 501, a fixed reference point 502, a set of decomposition tetrahedra 503, signed tetrahedron volumes 504, and a summation process 505 that yields the total enclosed volume.

In the illustrated embodiment, the volumetric computation proceeds by decomposing the oriented, watertight triangle mesh 501 into a set of signed tetrahedra 503 with respect to a fixed reference point 502 selected within or near the mesh, for example, a centroid to improve numerical conditioning. Let the vertices of an oriented triangle be a, b, and c, and let o denote the fixed reference point 502. The signed volume contributed by that triangle is

$$VT = \frac{1}{6} [ ( a - o ) \times ( b - o ) ] \cdot ( c - o ) .$$

Here, X denotes the vector cross product, which produces an oriented area vector perpendicular to the triangle plane, and ⋅ denotes the dot product, which projects that area vector onto the remaining edge to form the tetrahedron volume. The total enclosed volume is obtained by summing the per-triangle contributions over all faces in the mesh as indicated by the summation process 505,

$$V = {\sum }_{triangles} VT .$$

The sign of VT encodes triangle orientation: outward-facing normals produce positive contributions, inward-facing triangles produce negative contributions. A mesh that is watertight and consistently oriented is therefore essential for correctness. For numerical robustness, vertex coordinates may be re-centered so that o lies near the centroid of the model, which reduces cancellation in floating-point arithmetic. In practice, using double precision for the accumulation and performing explicit normal-direction checks for each face further stabilizes the result. From an intuitive standpoint, the method replaces the complex surface with many small pyramids whose tips meet at the reference point 502, then adds their signed volumes. For a closed and properly oriented triangle mesh, this approach is exact and avoids the discretization artifacts that arise when voxel grids are used.

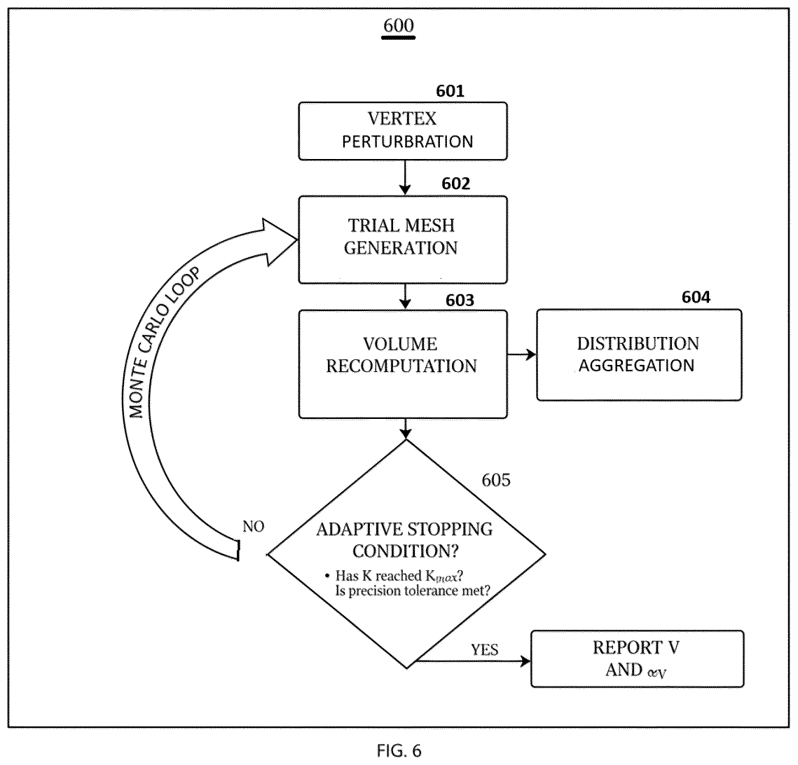

FIG. 6 illustrates a computational flow 600 demonstrating how vertex-level covariances are propagated to a scalar standard uncertainty σV. In general, the procedure models each vertex position as a random variable with a defined covariance, generates multiple realizations of the mesh consistent with those covariances, evaluates the enclosed volume for each realization, and computes the mean and standard deviation of the resulting volumes, denoted V and σV respectively. An adaptive stopping condition constrains the number of realizations such that the estimated uncertainty achieves a preselected precision level. The flow comprises vertex perturbation 601, trial mesh generation 602, volume recomputation 603, distribution aggregation 604, and an adaptive termination condition 605.

At element 601, each mesh vertex is treated as a random variable characterized by an estimated position and an associated covariance. For trial k, vertex j is perturbed according to

$${v}_{j , k} ∼ N ( μ j , \sum j ) .$$

The engine builds the trial mesh 602 using the same connectivity and orientation as the nominal mesh, computes the trial volume Vk at 603 via the signed-tetrahedra method of FIG. 5, and appends Vk to the set {Vₖ} at 604.

Estimators and convergence. After K trials, the device estimates V and σV as stated with FIG. 2. The stopping rule 605 controls K to reach a desired estimation precision without unnecessary computation. A practical criterion is

$$Stop when \frac{1.96 sV}{\sqrt{K}} \leq ε ,$$

where sV is the sample standard deviation of {Vₖ} and ε is a target half-width, for example two milliliters. This bounds the Monte Carlo induced noise in the reported mean. A first-order linear propagation can be used to sanity-check results by approximating

$$Var ( V ) \approx J Cov ( x ) {J}^{T}$$

where x stacks vertex coordinates and J is the gradient of V with respect to x. Because Vis cubic in coordinates, the Monte Carlo approach remains preferred for accuracy across a wide range of shapes and perturbations. Exemplary, configurable acquisition and sampling defaults are summarized in Table 1 below:

TABLE 1

| | | | | | | Reference |
|---|---|---|---|---|---|---|
| | | Coverage | | Stopping | | point |
| Device | | threshold, | Initial trials | tolerance, | Maximum | signed |
| class | Depth source | percent | K | milliliters | trials Kmax | tetrahedra |
| LiDAR | LiDAR | 95 | 5000 | 2.0 | 20000 | centroid |
| handset, | | (configurable) | (configurable) | (configurable) | (configurable) | |
| exemplary | | | | | | |
| RGB only | Simultaneous | 97 | 8000 | 2.0 | 30000 | centroid |
| handset, | Localization | (configurable) | (configurable) | (configurable) | (configurable) | |
| exemplary | and Mapping | | | | | |
| | with | | | | | |
| | anthropometric | | | | | |
| | scale | | | | | |

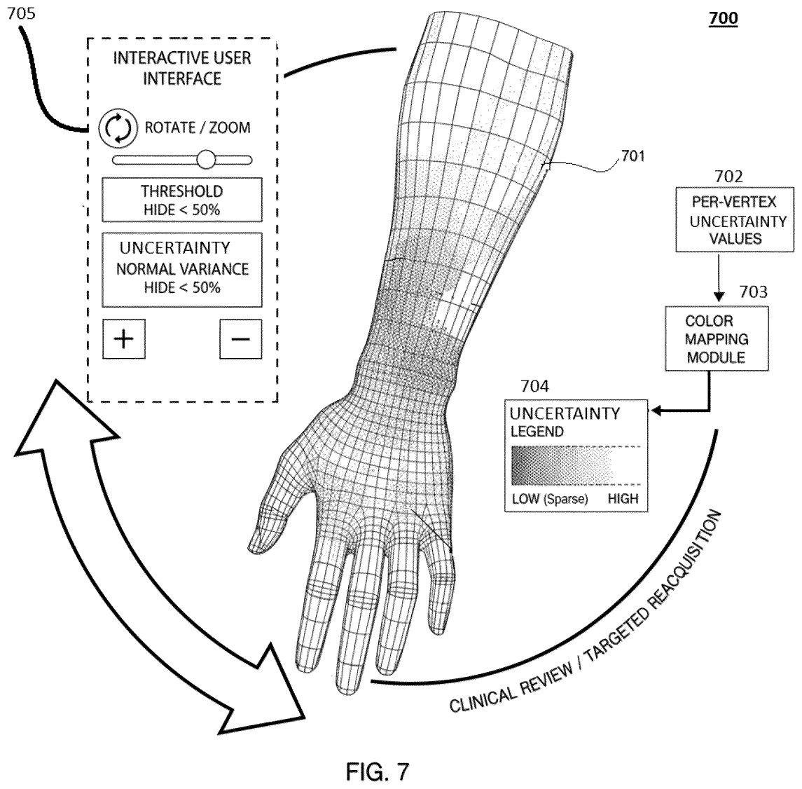

FIG. 7 is a diagram 700 illustrating a reconstructed region-of-interest mesh 701, per-vertex uncertainty values 702, a color-mapping module 703, an uncertainty heatmap 704, and an interactive user interface 705.

At element 702, the device computes a scalar uncertainty value for each vertex j derived from its associated covariance matrix Σj. In representative implementations, this scalar may correspond to the largest eigenvalue of Σj, which quantifies the maximum positional variance direction, or to an equivalent scalar function that characterizes overall positional uncertainty.

$$uj = λmax ( \sum j ) ,$$

or the predicted variance along the surface normal,

$$uj = {n}_{j}^{T} {\sum }_{j} {n}_{j} ,$$

where nj is the unit normal at vertex j. The color mapping module 703 applies a monotonic transfer function to obtain a display value,

$$cj = Clamp ( \frac{{u}_{j} - {u}_{min}}{{u}_{max} - {u}_{min}} , 0 , 1 ) ,$$

so that lower uncertainty maps near zero and higher uncertainty maps near one. The resulting field {cj} is rendered on the mesh 701 as the heatmap 704, with a legend indicating the correspondence between color and uncertainty magnitude. In embodiments intended for clinical review, the palette is chosen to remain interpretable by users with color vision deficiency, and values outside the selected range [uₘᵢₙ, uₘₐₓ] are clipped to avoid visual dominance by outliers.

At element 705, the interactive user interface permits rotation, zooming, and detailed inspection of the uncertainty heatmap 704, enabling a clinician or reviewer to visually distinguish surface regions that are well supported by data from those that warrant additional acquisition. The interface may further include a threshold control that selectively hides vertices whose confidence values cj fall below a user-selected threshold, a numeric readout displaying the uncertainty value uj at a cursor location, and a toggle control allowing the operator to switch between an eigenvalue-based uncertainty measure and a variance measure along the surface normal direction. When a scan has not yet satisfied the validation criteria described with reference to FIG. 2, the same heatmap is utilized to guide targeted reacquisition by indicating localized zones where additional views are predicted to most effectively reduce uncertainty.

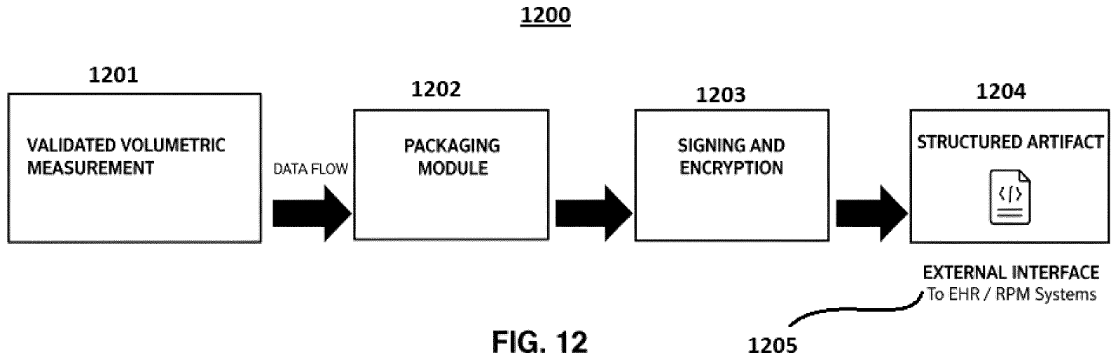

From a clinical and regulatory perspective, the uncertainty heatmap provides a transparent and auditable record of measurement quality. By explicitly displaying regions of high and low confidence, the system enables a reviewer to determine whether a reported volumetric value is supported by uniformly reliable geometric data or whether localized low-confidence regions persist. When the record exported in accordance with FIG. 12 includes either a compact representation of the heatmap or summary statistics derived from the set {uj}, downstream systems are capable of auditing not only the reported volume I′ but also the quality context in which that value was obtained. This enhances interpretability for longitudinal assessments by allowing a clinician to distinguish a true anatomical change from an apparent shift attributable to variations in coverage, alignment, or confidence between sessions.

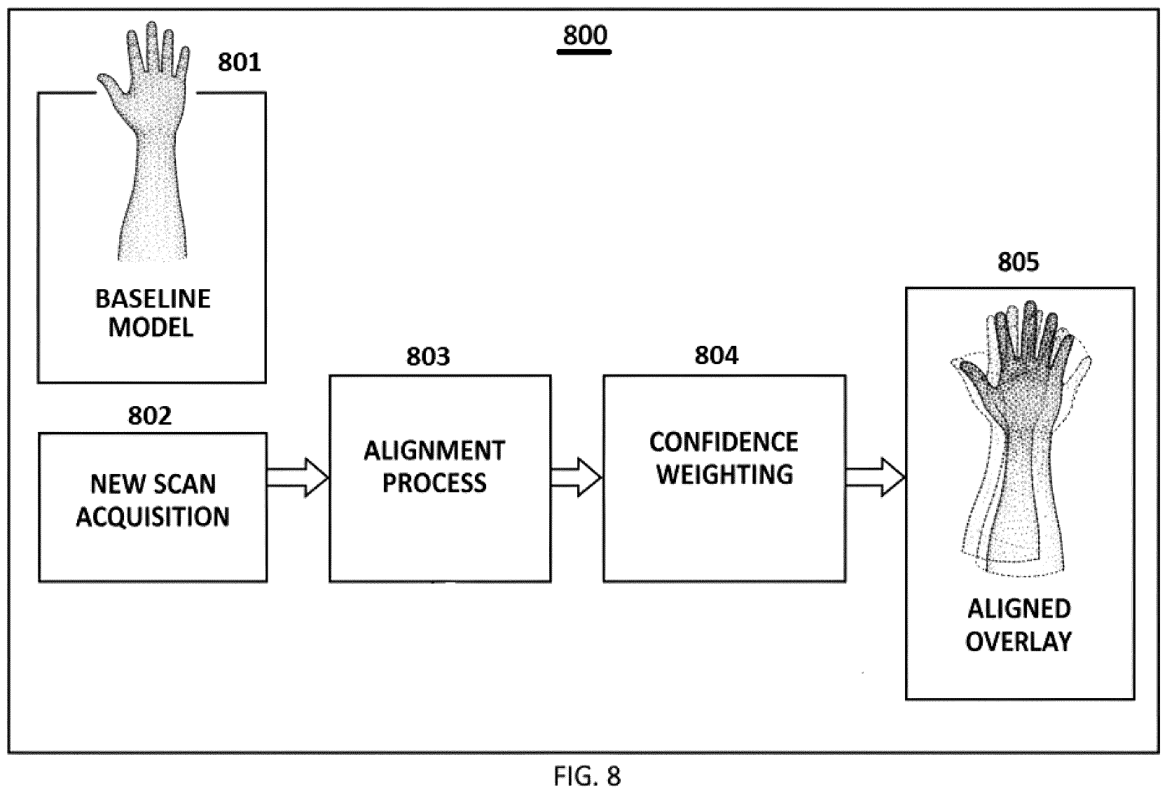

FIG. 8 is a diagram 800 illustrating a longitudinal analysis framework that includes storage of a baseline model 801, acquisition of a subsequent scan 802, an alignment process 803, confidence weighting 804, and an aligned overlay 805 used for temporal comparison.

At element 801, a high-quality baseline model for a given subject is captured under controlled conditions and stored as a persistent reference for future evaluations. At element 802, a follow-up scan of the same anatomical region is acquired. At element 803, the follow-up scan is registered to the baseline model. The registration may be rigid, estimating rotation and translation parameters, or deformable, permitting spatially varying displacements to accommodate posture differences and soft tissue changes between sessions.

At element 804, the registration objective is modulated by local confidence weighting so that regions of lower data quality in either scan contribute proportionally less to the alignment solution. In a representative deformable implementation, the optimization minimizes an objective function comprising a misfit term and a regularization term

$${min}_{ϕ} {\sum }_{x ∈ ROI} w ( x ) {‖ ϕ ( x ) - {x}_{baseline} ‖}_{2}^{2} + λ R ( ϕ ) ,$$

where ϕ maps points in the new scan 802 to the baseline 801, w(x) is a nonnegative weight derived from local uncertainty so that regions with higher uncertainty receive lower weight, and R(ϕ) is a regularizer, for example a smoothness penalty that discourages nonphysical deformations. In a rigid variant, ϕ(x)=Rx+t with rotation R and translation t, and the same weighting w(x) is applied to downweight low confidence correspondences.

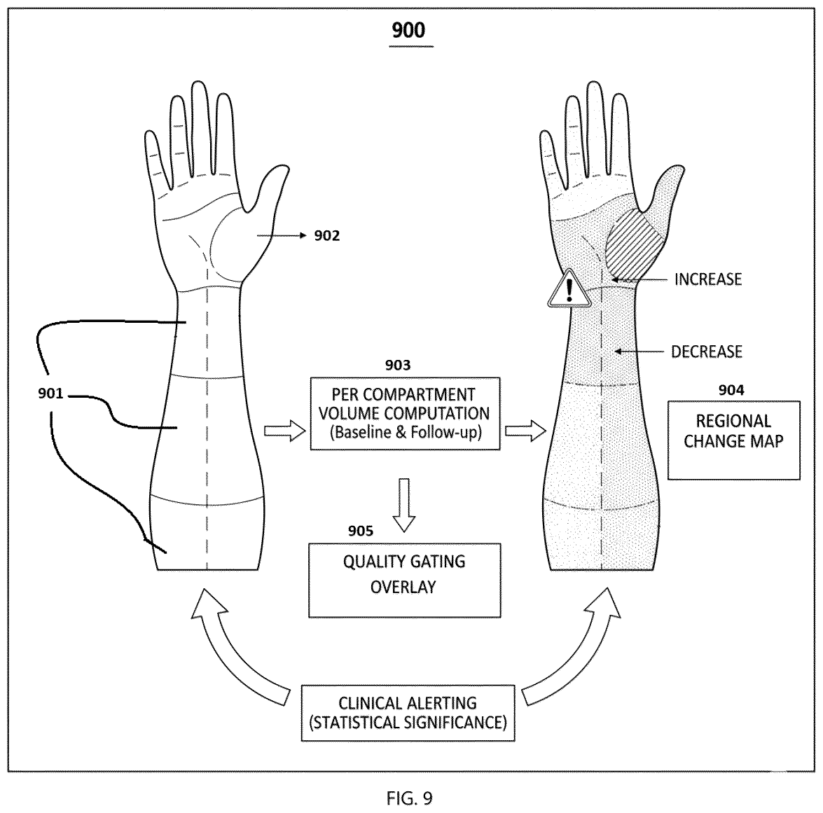

At element 805, the aligned overlay provides a visual and quantitative confirmation that the baseline and follow-up models have been accurately registered within a common spatial reference frame. This overlay enables subsequent regional change mapping and quality-weighted comparison as described with reference to FIG. 9, ensuring that volumetric differences are interpreted within a consistent coordinate context.

FIG. 9 is a diagram 900 illustrating a region-of-interest segmentation 901 into anatomical compartments 902, per-compartment volume computation 903, a regional change map 904, and a quality-gating overlay 905.

At element 901, the region of interest is partitioned into anatomically or clinically meaningful compartments 902. Representative partitions include foot, calf, and thigh for a lower limb, or hand, forearm, and upper arm for an upper limb. The system computes a volumetric measurement for each compartment at both the baseline and follow-up sessions, as indicated at 903, and then forms a compartment-wise volumetric difference that quantifies localized change over time.

$$Δ Vr = {V}_{r}^{( follow up )} - {V}_{r}^{( baseline )} ,$$

where the subscript r indexes the compartment.

At element 905, regions in which the local uncertainty exceeds a configured threshold are subject to gating within both the visualization and reporting layers. Such regions are either excluded from quantitative summaries or presented with an explicit caution indicator. The purpose of this gating mechanism is to prevent misinterpretation of a visually prominent but low-confidence region as a true physiological or clinical change when the underlying data fidelity is inadequate to support such an inference.

At element 904, the device generates a regional change map that color-codes the compartmental volume differences, denoted ΔVr, to facilitate intuitive interpretation. In one representative convention, decreases in regional volume are displayed in one hue family, increases in another, and regions gated for insufficient confidence are rendered in a neutral gray tone. This presentation enables a reviewer to visually localize the distribution of change-such as distinguishing a reduction localized to the ankle from one involving the calf rather than relying solely on an aggregate global metric.

For clinical alerting and quantitative validation, the device computes a standard uncertainty of the regional change, denoted σΔV, and applies a decision rule requiring that the absolute change magnitude exceed a prescribed multiple of that uncertainty before a region is flagged as significant:

$$\vert Δ V \vert > k σΔ V ,$$

with the factor k set by clinical policy, for example three. When two validated scans are treated as independent, the change uncertainty is

$$σΔ V = \sqrt{{σ}_{V 1}^{2} + {σ}_{V 2}^{2}} ,$$

If a shared bias or correlation exists between the baseline and follow-up scans, a covariance term may be incorporated into the uncertainty propagation so that

$$σΔ V = \sqrt{{σ}_{V 1}^{2} + {σ}_{V 2}^{2} - 2 Cov ( V 1 , V 2 )}$$

where Cov (V₁, V₂) denotes the covariance between the two volume estimates. Depending on the sign of this covariance, the resulting uncertainty of the change may be reduced or increased accordingly. Incorporating this covariance-aware formulation refines the statistical interpretation of longitudinal changes, mitigates false-positive detections arising from random variation, and ensures that reported differences reflect changes supported by measurable evidence rather than stochastic noise.

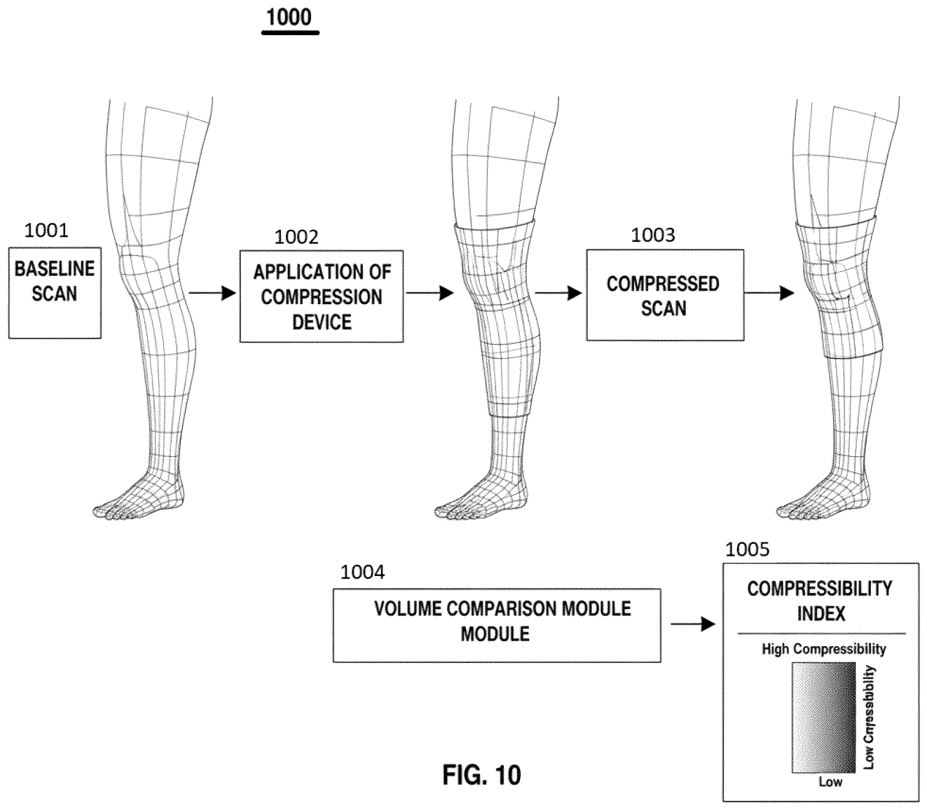

FIG. 10 is a diagram 1000 illustrating a two-scan protocol comprising a baseline scan 1001, application of a compression device 1002 that exerts a controlled and quantifiable pressure, a compressed scan 1003, a volume comparison module 1004, and a derived compressibility index 1005.

At element 1001, the device acquires a baseline volumetric measurement under resting physiological conditions. At element 1002, an external compression device applies a known pressure, for example, approximately thirty millimeters of mercury, to the anatomical region of interest. With the limb or region stabilized to minimize motion, the operator performs a second scan at element 1003. The processing module at element 1004 computes the volumetric change and its corresponding uncertainty, incorporating covariance terms when appropriate to provide a statistically defensible compressibility index.

$$Δ V = {V}_{baseline} - {V}_{compressed} , σΔ V = \sqrt{{σ}_{Vbaseline}^{2} + {σ}_{Vcompressed}^{2}} ,$$

assuming independence between scans. If shared bias is present, a covariance term may be included.

At element 1005, the device reports a compressibility index that expresses the percentage reduction in volume at the applied pressure,

$$CI ( \% ) = 100 \times \frac{Vbaseline - Vcompressed}{Vbaseline} ,$$

together with an associated uncertainty derived from σΔV. Larger values of the compressibility index can indicate fluid dominant tissue that is more pressure responsive, while smaller values can indicate fibrotic tissue that is less responsive, which complements baseline change in volume trends. The same acceptance gates are applied to both scans so that the comparison reflects measurements of commensurate quality. If either scan fails a coverage, alignment, blur, confidence, or uncertainty threshold, export is inhibited until the deficiency is remedied. This prevents spurious compressibility values that could otherwise arise from inadequate data fidelity.

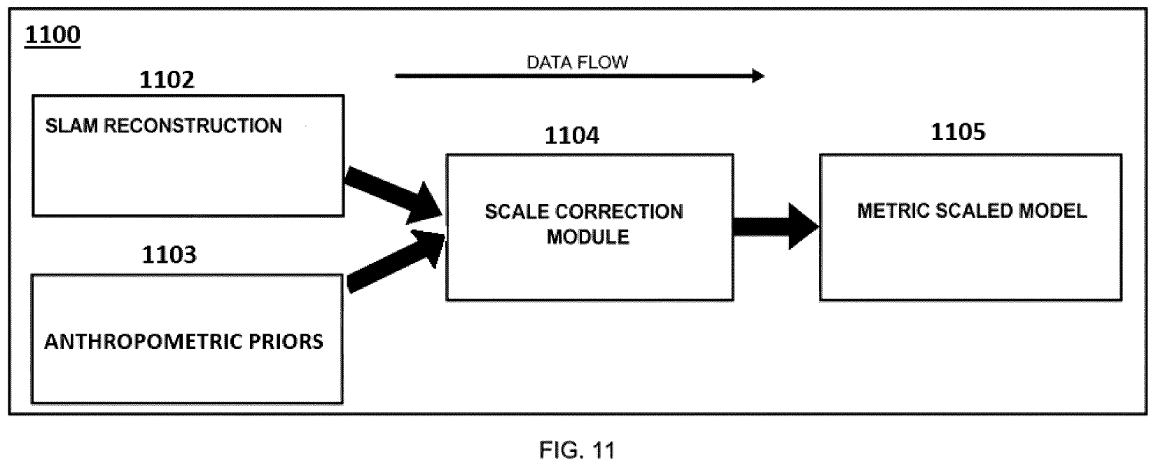

FIG. 11 is a diagram 1100 illustrating a mobile device 1101 employing Simultaneous Localization and Mapping reconstruction 1102, anthropometric priors 1103, a scale correction module 1104, and a resulting metric-scaled model 1105.

At element 1102, the device generates an initial three-dimensional reconstruction using Simultaneous Localization and Mapping. On devices lacking a hardware depth sensor, a monocular reconstruction pipeline may drift in absolute scale, yielding geometry that is correct in shape yet biased in size. Because volume scales with the cube of linear scale, even a modest scale bias can induce a substantially larger volumetric error.

At element 1103, the device introduces anthropometric priors in the form of soft constraints on plausible lengths and circumferences for the anatomy being scanned. These priors may include ranges for limb-segment lengths and circumference bands at anatomical landmarks, together with tolerances that reflect natural population variability.

At element 1104, the device combines the Simultaneous Localization and Mapping reconstruction with the anthropometric priors in a constrained optimization that refines the global metric scale. In a representative formulation, the objective minimizes a weighted misfit between model-implied landmark distances and the anthropometric priors, together with a regularization term that preserves the relative shape recovered by Simultaneous Localization and Mapping, subject to bounds derived from measurement tolerances. The solution yields a single scale factor or a small set of scale parameters that, when applied to the reconstruction, produce the metric-scaled model 1105 suitable for volumetric computation and downstream clinical use.

$${min}_{s > 0} {\sum }_{m} ρ ( \frac{{M}_{m} ( s ) - {M}_{m}^{prior}}{{σ}_{m}} ) ,$$

where s is the unknown global scale factor, Mm(s) are model derived measurements under scale s such as circumference at specified landmarks,

$${M}_{m}^{prior}$$

are prior targets with tolerances σₘ and ρ(˜) is a robust penalty that reduces the influence of outliers. The initial scale may be seeded by visual and inertial Simultaneous Localization and Mapping, in which inertial data are provided by an Inertial Measurement Unit.

At element 1105, the optimized scale factor is applied to the reconstructed model to produce a metrically scaled representation whose linear dimensions and volumetric values are expressed in physical units suitable for clinical computation and longitudinal analysis. This approach corrects absolute scale without requiring fiducial markers, preserves the intrinsic surface geometry of the reconstruction, and stabilizes volumetric measurements on devices that rely solely on monocular sensing.

FIG. 12 is a diagram 1200 depicting assembly of a validated volumetric measurement 1201, a packaging module 1202, a signing and encryption process 1203, a structured artifact 1204, and an external interface 1205 configured for communication with Electronic Health Record systems and Remote Patient Monitoring platforms.

At element 1201, the validated measurement includes, at a minimum, the estimated volume V, the associated standard uncertainty σV, and a set of per-scan Quality Assurance metrics comprising surface coverage percentage, alignment error expressed as a root mean square residual, a motion blur index, and indicators specifying which validation thresholds were met. The record may further include selected visualization summaries such as a reduced uncertainty heatmap thumbnail or statistical descriptors derived from the per-vertex uncertainty field. Device and software provenance data are incorporated to document the conditions under which the measurement was acquired, validated, and approved for export.

At element 1202, the packaging module serializes these data elements into a structured artifact 1204 suitable for interoperable system-to-system exchange. In representative embodiments, the artifact is encoded in JavaScript Object Notation (JSON) or Extensible Markup Language (XML) and may conform to an established healthcare interoperability framework, for example, a Fast Healthcare Interoperability Resources (FHIR) resource type. This standards-based representation allows receiving systems to automatically parse and integrate the measurement without custom implementation. An exemplary, non-limiting list of structured export fields is presented in Table 2 below.

TABLE 2

| Field name | Type | Required | Description |
|---|---|---|---|
| Volume V | milliliters | required | Estimated enclosed volume computed on |
| | | | the validated mesh |
| Standard uncertainty | milliliters | required | Standard uncertainty of V derived from |
| σV | | | the sampling procedure |
| Coverage percent | percent | required | Surface coverage over the region of |
| | | | interest |
| Alignment root | millimeters | required | Root mean square point to surface |
| mean square | | | residual |
| Blur index | unitless | optional | Device specific motion or sharpness |
| | | | metric |
| Thresholds met | boolean or list | required | Indicators of acceptance gates satisfied |
| Heatmap summary | string or small image | optional | Summary of uncertainty distribution or |
| | reference | | link to artifact |
| Device model | string | required | Hardware identifier of the mobile device |
| Software build | string | required | Version of capture and processing |
| | | | software |
| Digital signature | string | required | Signature for integrity and origin |
| | | | verification |
| Encryption flag | boolean | required | Indicates whether artifact is encrypted in |
| | | | transit and at rest |
| Timestamp | ISO 8601 | required | Time of capture and validation |
| Patient or subject | tokenized string | optional | Pseudonymized identifier consistent with |
| identifier | | | privacy policy |

At element 1203, authenticity and privacy safeguards are applied to ensure data integrity, confidentiality, and regulatory compliance. A cryptographically verifiable digital signature is attached to the artifact so that recipients can confirm both the integrity of the content and its origin. Encryption is applied to protect data in transit and at rest, thereby preserving patient privacy and satisfying applicable clinical, institutional, and regulatory requirements for secure handling of protected health information.

At element 1205, the structured artifact 1204 is transmitted to an Electronic Health Record (EHR) system or to a Remote Patient Monitoring (RPM) platform. In longitudinal operation, the receiving system may evaluate clinical alerts based on a change detection rule that compares the absolute magnitude of a measured change to a multiple of the corresponding uncertainty:

$$\vert Δ V \vert > k \cdot σ\_Δ V ,$$

where k is a configurable factor established by clinical policy. Because the artifact includes detailed Quality Assurance metrics and indicators specifying which acceptance gates were satisfied, downstream systems can associate any alert or interpretation with the quantitative measurement quality that underlies it.

For auditability and traceability, the artifact incorporates acceptance indicators corresponding to the gating and validation processes described with reference to FIG. 2, as well as environmental and device provenance data. These elements enable retrospective verification, regulatory audit, and independent reproduction of measurement conditions. This structured packaging architecture preserves the explicit linkage between the reported value I′, its associated uncertainty σV, and the quantitative Quality Assurance context in which the measurement was produced. The result is a verifiable, interoperable record that supports reliable clinical decision-making and defensible longitudinal comparison.

The following examples describe representative implementations of the disclosed systems and methods. Unless otherwise noted, the system executes on a single mobile handset equipped with a color image sensor, a depth-sensing capability, and an inertial measurement unit.

### Example of Upper Limb, Best Mode, Longitudinal Alert

The region of interest was an upper limb extending from the wrist crease to the mid upper arm. Acceptance thresholds were: surface coverage greater than or equal to ninety five percent, alignment residual root mean square less than or equal to 0.9 millimeters, blur index less than or equal to 0.25, absolute uncertainty gate σV less than or equal to 42 milliliters, and relative uncertainty gate σV/V less than or equal to 1.7 percent. The longitudinal significance factor k was three. The subject's forearm was scanned in a seated position with the limb supported. The device to subject distance was maintained between 0.45 meters and 0.75 meters. Next best view cues were followed until the coverage meter reached greater than or equal to ninety five percent. Baseline yielded V1=2,500 milliliters with σV1=14 milliliters after K≈8,192 Monte Carlo trials, with a Monte Carlo half width on V less than or equal to two milliliters. A follow up scan after seven days yielded V2=2,565 milliliters with σV2=15 milliliters after K≈10,000 trials. Gates were satisfied for both scans. The change was ΔV=+65 milliliters with σΔV=√(14\hat{ }2+15\hat{ }2)≈20.5 milliliters. The alert condition |ΔV|>3·σΔV was satisfied, since 65>61.5, and an alert was generated. The export bundle contained V, σV, the scan quality metrics, and indicators of thresholds satisfied.

### Example of Lower Limb, Compression Under Load, Compressibility Index

The region of interest was a lower limb from malleoli to mid thigh. Acceptance thresholds were: coverage greater than or equal to ninety four percent, alignment residual root mean square less than or equal to 1.1 millimeters, blur index less than or equal to 0.30, absolute uncertainty gate σV less than or equal to 85 milliliters, and relative uncertainty gate σV/V less than or equal to 2.0 percent. A pneumatic sleeve applied a target pressure of thirty millimeters of mercury. The longitudinal significance factor k was three. In all working examples, the divergence theorem volume was computed by summing signed tetrahedra with respect to a reference point near the mesh centroid. Vertices were recentered to improve numerical conditioning, and double precision accumulation was used. Monte Carlo trials perturbed each vertex as vj,k˜N(μj, τj). The adaptive stopping rule increased K until the half width of the selected confidence interval on V or on σV was less than or equal to a configured tolerance. A resting scan yielded Vbaseline=3,400 milliliters with σVbaseline=25 milliliters. With the sleeve pressurized and the limb immobilized, a compressed scan yielded Vcompressed=3,280 milliliters with σVcompressed=28 milliliters. Gates were satisfied for both scans. The change was ΔV=120 milliliters with σΔV≈√(25\hat{ }2+28\hat{ }2)≈37.6 milliliters, so |ΔV|>3·σΔV(120>112.7) was satisfied. The compressibility index was CI=100×(Vbaseline−Vcompressed)/Vbaseline≈3.53 percent with uncertainty approximately 100×σΔV/Vbaseline≈1.11 percent. The device reported the index and its uncertainty together with the validated scan metrics.

### Example of Phantom Validation Against Predicted Uncertainty

A solid sphere phantom of diameter 160 millimeters was scanned thirty times under controlled lighting. The reference volume by caliper measurement was Vref≈2,144.66 milliliters. For each repetition, the device produced V and σV using the same mesh and Monte Carlo procedure. The predicted per scan uncertainty averaged σV=12 milliliters. The empirical standard deviation across the thirty reconstructed volumes was sV=13 milliliters. The acceptance criterion |sV/σV−1| less than or equal to 0.2 was met (13/12≈1.08). The mean reconstructed volume differed from Vref by less than 0.2 percent. The validation log was stored as part of the device quality records.

### Example of Back to Back Repeatability Gate

The two scan repeatability gate required an absolute difference less than or equal to 2·σΔV. Two scans acquired within five minutes yielded V_A=2,508 milliliters with σV_A=16 milliliters and V_B=2,520 milliliters with σV_B=17 milliliters. The observed difference was |ΔV]=12 milliliters with σΔV=√(16\hat{ }2+17\hat{ }2)≈23.3 milliliters. The acceptance criterion 12≤2.23.3 was satisfied, and export remained enabled for both scans.

### Example of Export Lockout with Targeted Rescan Guided by Uncertainty Heatmap

An upper limb scan initially reached coverage 88 percent, alignment residual root mean square 1.4 millimeters, blur index 0.33, and predicted σV=68 milliliters. Export was blocked because coverage and σV gates were not satisfied. The device displayed an uncertainty heatmap showing elevated uncertainty on the posterior forearm and provided next best view prompts. After forty seconds of guided motion, the updated model reached coverage 96 percent, alignment residual root mean square 0.82 millimeters, blur index 0.19, and σV=18 milliliters. All gates were satisfied, at which point the device computed V=2,486 milliliters and enabled export. The export bundle included a thumbnail of the uncertainty heatmap and the quantitative metrics.

### Example of Markerless Metric Scaling on a Smartphone without Hardware Depth

A monocular Simultaneous Localization and Mapping pipeline produced an initial reconstruction of a mid calf region. Anthropometric priors specified an expected mid calf circumference of 360 millimeters with a tolerance of ±10 millimeters. The scaling optimization minimized the discrepancy between model derived circumferences and prior targets, subject to a positive global scale. The unscaled model circumference at mid calf was 335 millimeters. The optimization yielded s≈1.0746, bringing the model circumference to 360 millimeters within the tolerance. The raw model volume for the region of interest was 2,800 milliliters; the scaled volume was s\hat{ }3×2,800≈3,475 milliliters. The scaled uncertainty was s\hat{ }3×σV_raw. The scaled scan then satisfied σV and relative uncertainty gates and was exported with provenance indicating the applied scale factor and the prior set used.

### Example of Adaptive Monte Carlo Stopping and Parallel Execution

The device targeted a Monte Carlo induced half width on V less than or equal to two milliliters at approximately ninety five percent confidence. Trials were executed in parallel on a graphics processor. For a representative upper limb region V≈2,430 milliliters, the adaptive controller increased K as follows: K=1,024, estimated half width≈3.9 milliliters; K=2,048, ≈2.8 milliliters; K=4,096, ≈2.3 milliliters; K=8,192, ≈1.6 milliliters. The process stopped at K=8,192. A cross check using a first order linear propagation produced a variance estimate within ten percent of the Monte Carlo estimate. The recorded σV was taken from the Monte Carlo distribution.

### Example of Baseline Anchoring, Confidence Weighted Registration, and Regional Delta Volumes

A high quality baseline model of a lower limb was stored. The follow up scan used confidence weighted deformable registration to the baseline. A compartmental segmentation was applied with foot, calf, and thigh regions. Regions with local uncertainty exceeding a configured limit were gated in visualization. Baseline compartment volumes were: foot 350±7 milliliters, calf 1,200±22 milliliters, thigh 2,100±36 milliliters. Follow up volumes were: foot 360±8 milliliters, calf 1,250±24 milliliters, thigh 2,090±40 milliliters. Compartment changes were ΔV_foot=+10 milliliters with σΔV≈10.6 milliliters, ΔV_calf=+50 milliliters with σΔV≈32.6 milliliters, and ΔV_thigh=−10 milliliters with σΔV≈53.8 milliliters. No compartment satisfied |ΔV_r|>3·σΔV_r, so no regional alert was issued. The overlay displayed the regional change map with a neutral tone applied to a posterior calf subregion that had been gated due to elevated local uncertainty.

### Example of Acceptance Gates Tied to Clinical Minimal Meaningful Change

Clinic policy specified minimal clinically meaningful change (MMC_abs, MMC_rel) of 125 milliliters and five percent for the upper limb and 250 milliliters and five percent for the lower limb. The significance factor k was three, so the absolute σV gate was MMC_abs/k, and the relative σV/V gate was MMC_rel/k. An upper limb scan with V=2,480 milliliters and σV=40 milliliters satisfied the absolute gate 40≤41.7 and the relative gate 40/2,480≈1.61 percent≤1.67 percent, so export was enabled. A lower limb scan with V=4,960 milliliters and σV=92 milliliters failed the absolute gate 92≤83.3 and was blocked. The device displayed the uncertainty heatmap and next best view cues to reduce σV below the gate.

### Example of Confidence Weighted Alignment Reduces Pose Bias

Two scans of the same limb were acquired with slight motion artifacts in one scan localized to the distal calf. Rigid registration to a baseline was solved twice: once with uniform weights and once with per vertex weights derived from the largest eigenvalue of Σj. Uniform weighted alignment produced a mean surface misfit of 1.2 millimeters and a location dependent bias that increased ΔV estimates by approximately 35 milliliters in the distal calf. Confidence weighted alignment reduced mean misfit to 0.8 millimeters and reduced the distal calf bias to less than 10 milliliters.

### Example of Failure Mode and Corrective Behavior

A home capture was attempted in low light with a patterned background. The system detected a blur index of 0.41 and an alignment residual root mean square of 1.9 millimeters during acquisition. Export remained locked, and the interface instructed the user to increase lighting and to step back by fifteen centimeters. After corrective steps, coverage reached ninety six percent, the blur index dropped to 0.21, alignment residual root mean square dropped to 0.88 millimeters, and σV decreased from 75 milliliters to 22 milliliters. The scan then satisfied all gates and was exported.

### Example of Report Packaging and Provenance for Clinical Systems

The device compiled a structured artifact that conformed to a healthcare interoperability profile and included digital signing and optional encryption. For the follow up scan, the artifact contained: patient and encounter identifiers, device make and software version, V=2,565 milliliters, σV=15 milliliters, a two sided ninety five percent confidence interval derived from σV, coverage 96 percent, alignment residual root mean square 0.78 millimeters, blur index 0.18, the significance factor k=3 used for alerting, and acceptance indicators for all gates. The artifact was digitally signed on device and transmitted to a remote patient monitoring endpoint. An audit record of the Monte Carlo trial count and stopping criterion was included.

### Example of Loop-Closure Drift Detection and Recovery

A circular capture of a lower limb from malleoli to mid thigh was performed to evaluate drift over a closed path. Acceptance thresholds included coverage greater than or equal to ninety five percent, alignment residual root mean square less than or equal to 1.0 millimeters, blur index less than or equal to 0.25, absolute uncertainty gate V less than or equal to 60 milliliters, and loop-closure drift less than or equal to 1.5 millimeters between start and end poses. The initial pass reached coverage 95 percent, alignment residual root mean square 0.96 millimeters, blur index 0.22, σV=24 milliliters, but loop-closure drift measured 2.3 millimeters. Export was blocked. The device instructed the user to revisit the starting sector and hold steady for two seconds, then to re-trace the final arc. After reacquisition, loop-closure drift decreased to 0.9 millimeters with coverage 96 percent and σV=19 milliliters. All gates were satisfied and export was enabled with loop-closure diagnostics recorded in the audit bundle.

### Example of Analytic Uncertainty Propagation Reduces Samples

For an upper limb region of interest with acceptance thresholds, the analytic Bayesian uncertainty engine was enabled alongside Monte Carlo sampling. With the analytic engine, the device reported V=2,515 milliliters and σV_analytic=16 milliliters using a closed-form propagation. A verification Monte Carlo run with K=2,048 trials produced V=2,513 milliliters and σV_MC=17 milliliters, meeting the target half width on V of less than or equal to two milliliters without increasing K to 8,192. Gates tied to σV (absolute 42 milliliters and relative 1.7 percent) were satisfied. The audit bundle recorded both estimators and their agreement within five percent, demonstrating reduced computational load while preserving acceptance rigor.

### Example of Enforced Dwell on a Missing Sector

An upper limb scan initially achieved coverage 93 percent with a contiguous low-confidence patch on the posterior forearm measuring approximately twenty five square centimeters. Acceptance thresholds required coverage greater than or equal to ninety five percent and a dwell of at least two seconds on any missing sector flagged by the coverage tracker. Export was blocked. The interface directed the user to position the device at 0.55 meters and hold steady on the highlighted sector. After 2.5 seconds of dwell, the sector transitioned from red to green, overall coverage increased to 96 percent, alignment residual root mean square improved from 1.05 millimeters to 0.84 millimeters, and σV decreased from 34 milliliters to 18 milliliters. All gates were satisfied and export proceeded, with the dwell satisfaction recorded in the audit log.

### Example of Lymphedema Monitoring for Early Swelling Detection

A breast cancer survivor's arm was scanned to establish a baseline volume after surgery. A follow-up scan six weeks later under similar conditions yielded a volume increase of 4.0% (approximately 120 mL). All quality gates were satisfied for both scans. The observed change exceeded the significance threshold (|ΔV|>3·σΔV), triggering an early alert. In clinical context, arm heaviness and swelling symptoms typically begin when volume increases about 5-10%, and clinical diagnosis often requires a >10% limb volume increase. In this example, the system detected a ˜4% volume change, within the asymptomatic range, illustrating how at-home 3D scanning can catch subclinical lymphedema earlier than traditional methods. Early detection enables timely intervention (for example, compression therapy), aligning with prospective surveillance models that seek to identify lymphedema at only 3-5% volume increase. The exported audit bundle for each scan documented that coverage, alignment, blur, and uncertainty criteria were met, ensuring the volume change was measured with high confidence.

### Example of Lipedema Assessment with Segmental Volume and Compressibility

A patient with lipedema in the lower limbs underwent baseline and post-compression scans to quantify adipose-related volume and tissue stiffness. The region of interest was segmented into thigh and calf compartments. Baseline volumes were: calf 3,200 mL and thigh 5,100 mL. A pneumatic compression sleeve at 30 mmHg was applied for 5 minutes, then an immediate follow-up scan was acquired. Post-compression volumes were: calf 3,110 mL and thigh 5,060 mL, corresponding to modest reductions of 2.8% and 0.8% respectively. All scans passed coverage (≥95%) and uncertainty gates (σV<2% of volume). The compressibility index for the calf was 2.8%, substantially lower than the ˜3.5% index observed in pure fluid edema under similar conditions under similar compression conditions. This finding is consistent with lipedema's fibrotic adipose tissue, which responds less to short-term compression than fluid-dominant edema. The example demonstrates that segmental 3D volumetry can quantify volume changes in lipedema and potentially distinguish fat-dominant swelling from fluid accumulation. The system's uncertainty-aware metrics ensured that even these small volume changes (on the order of tens of milliliters) were above the noise level of the measurement. Each validated scan was packaged with its segmental volumes and an audit record, providing an objective baseline for tracking lipedema progression or therapy response over time.

### Example of Remote Edema Monitoring in Heart Failure

A congestive heart failure patient used the mobile scanning system daily to monitor ankle and lower-leg edema. At hospital discharge, a baseline volume of the ankle region (malleolus to mid-calf) was recorded as 1,650 mL (σV=10 mL). Over two weeks of home monitoring, volumes remained within ±1.5% of baseline until day 15, when the volume measured 1,740 mL (a ±5.5% increase from baseline). The increase persisted on day 16. All scans met quality acceptance criteria (for example, alignment RMS<1 mm, coverage>90%). Given the system's uncertainty (σΔV=14 mL), the volume gain was statistically significant and exceeded the clinical alert threshold. This outcome correlates with heart failure decompensation, where approximately 70% of acute heart failure hospitalizations present with peripheral edema, and ankle circumference or volume is a reliable gauge of fluid retention. The system issued an alert to the care team, allowing intervention (diuretic adjustment) before the patient reached a crisis. Continuous or frequent 3D volumetric scanning in such patients provides quantitative tracking of fluid status, akin to an “edema vital sign.” Early detection of edema trends can trigger timely therapy adjustments and potentially avert hospitalizations. The example illustrates how the invention supports remote patient monitoring: each scan's digitally signed report (including volume, σV, and quality indicators) was transmitted to an electronic health record. Clinicians reviewing the trend had confidence in the data integrity and could act on the clear upward deviation, rather than relying on subjective swelling reports or weight alone (weight changes often lag or misrepresent fluid changes). This examiner-friendly scenario underscores the system's value in managing chronic heart failure by providing objective, repeatable edema measurements in home settings.

### Example of Wound Volume Tracking in Chronic Wound Care

The system was applied to measure and track the volume of a chronic venous ulcer on a patient's lower leg. A region of interest around the wound was scanned weekly. The baseline scan produced a wound volume of 6.2 mL (ulcer cavity and undermined area) with σV=0.2 mL. The mesh was watertight after automatic hole-filling of the wound opening. Over four weeks, the wound's volume decreased to 4.5 mL, then 3.1 mL, and eventually 1.0 mL as healing progressed (each measurement with σV under 0.3 mL). Traditional wound measurement techniques (rulers for length or width, or tracing) cannot reliably capture volume and often yield inconsistent area estimates. In contrast, the 3D scanning approach provided accurate point-of-care data on wound dimensions and volume, supporting objective monitoring of healing. All scans were acquired with real-time quality guidance to ensure the camera captured the wound from multiple angles at adequate range, avoiding occlusions. For instance, the system prompted the user to angle the device for better side-wall coverage of the ulcer, turning the live mesh green in previously red (unseen) areas once sufficient data was captured. Each accepted scan generated a report with the wound's volume and a color-coded uncertainty map. Clinicians used these outputs to adjust treatment, such as noting a plateau in volume reduction between weeks 2 and 3, leading to debridement of devitalized tissue. This example highlights the invention's utility beyond limb volumetry: by enabling wound volume tracking over time, it aids in treatment planning and outcome documentation. The high-resolution 3D data (sub-millimeter accuracy) ensured that even subtle changes in wound depth were measured. All measurements remained non-invasive and contact-free, important for infection control and patient comfort. The system's closed-loop guidance and quantitative thresholds were examiner-friendly, demonstrating clear superiority over subjective or 2D methods in a manner supported by current wound care research, which emphasizes 3D measurement for more reliable assessment.

1. A computer-implemented method executed on a mobile device for generating a validated three-dimensional model of an anatomical region, the method comprising:
(a) capturing, via at least one camera and a three-dimensional sensing capability of the mobile device, a stream of data of the anatomical region;
(b) generating, by a processor of the mobile device during the capturing, a three-dimensional surface model of the anatomical region from the stream of data, the surface model comprising a plurality of vertices, and associating, for vertices or surface elements of the model, location-specific position uncertainty values;
(c) computing, by the processor during the capturing, one or more quantitative scan-quality metrics including at least one metric based on the positional uncertainty values;
(d) providing, via a display of the mobile device, real-time corrective guidance to a user, wherein the guidance is determined by a spatial distribution of the positional uncertainty values over the surface model and is configured to direct user motion to acquire additional data that reduces the positional uncertainty values; and
(e) preventing export of the three-dimensional surface model or any measurement derived therefrom from the mobile device when at least one of the one or more scan-quality metrics fails to satisfy a predefined validation threshold, and enabling export only when all predefined validation thresholds are satisfied.

2. The method of claim 1, wherein the one or more scan-quality metrics further comprise at least one metric selected from the group consisting of a surface coverage percentage, a motion-induced blur level, and a root-mean-square frame-to-model alignment residual.

3. The method of claim 1, wherein providing the real-time corrective guidance comprises displaying an uncertainty heatmap on a rendering of the surface model and displaying augmented-reality overlays indicating a next best view.

4. The method of claim 1, wherein the predefined validation thresholds include a maximum positional-uncertainty threshold for the surface model.

5. The method of claim 1, wherein the positional uncertainty value is a three-by-three position covariance matrix based on at least one of sensor-reported depth confidence, viewing range, or surface incidence angle, and wherein the method further comprises fusing position covariance matrices from multiple distinct views of the same surface location by summing information matrices.

6. The method of claim 5, wherein generating the three-dimensional surface model comprises performing a confidence-weighted registration that uses the position covariance matrices to weight contributions of different portions of the surface model to an alignment objective.

7. The method of claim 1, wherein the predefined validation thresholds comprise a minimum global coverage percentage and a maximum contiguous uncovered area within the region of interest.

8. The method of claim 1, further comprising enforcing operational guardrails, including pausing capture when range or incidence constraints are violated for a sustained fraction of frames and halting capture upon tracking resets or drift beyond a threshold.

9. The method of claim 1, further comprising measuring a volume of the anatomical region with quantified uncertainty, the measuring comprising:
(a) processing the three-dimensional surface model to be watertight and consistently oriented;
(b) computing a volumetric measurement from the watertight and consistently oriented surface model;
(c) propagating the positional uncertainty values through a volume computation function to generate a standard uncertainty of the volume; and
(d) making a determination based on the standard uncertainty of the volume, the determination selected from the group consisting of:
(i) enabling export of the volumetric measurement only if the standard uncertainty satisfies a predefined precision threshold; and
(ii) generating a clinical alert for a change in volume over time relative to a baseline model only if an absolute magnitude of the change exceeds a configurable multiple of a propagated uncertainty of the change, the propagated uncertainty accounting for covariance between measurements when applicable.

10. The method of claim 9, wherein the positional uncertainty values comprise position covariance matrices, and wherein propagating the position covariance matrices comprises performing a Monte Carlo simulation wherein, for each trial, each vertex is perturbed according to its associated position covariance matrix to generate a trial mesh, and a trial volume is computed from the trial mesh, and wherein a number of trials for the Monte Carlo simulation is adaptively increased until a confidence-interval half-width for the volumetric measurement is at or below a predefined tolerance.

11. The method of claim 9, wherein propagating the positional uncertainty values comprises analytic probabilistic propagation that estimates a mean and a variance of volume from a probabilistic model of surface geometry and sensor noise.

12. The method of claim 9, wherein computing the volumetric measurement comprises performing a divergence-theorem summation of signed tetrahedron volumes relative to a reference point.

13. The method of claim 9, wherein the predefined precision threshold comprises an absolute precision gate and a relative precision gate derived from a clinic-defined minimal clinically meaningful change.

14. The method of claim 9, wherein the determination comprises generating the clinical alert, and wherein the method further comprises: storing the baseline model; registering the three-dimensional surface model to the baseline model using a confidence-weighted registration; and computing the change in volume and the propagated uncertainty of the change.

15. The method of claim 1, wherein the three-dimensional sensing capability is provided by a camera and a visual-inertial simultaneous localization and mapping process, and wherein the method further comprises stabilizing an absolute metric scale of the model by fusing a scale derived from the process with one or more anthropometric priors.

16. A mobile device system for generating a validated three-dimensional model of an anatomical region, the system comprising:
(a) at least one camera and a three-dimensional sensing capability;
(b) a display;
(c) a processor; and
(d) a non-transitory memory storing instructions that, when executed by the processor, configure the mobile device to:
(i) capture a stream of data of the anatomical region;
(ii) generate a three-dimensional surface model of the anatomical region, and associate, for vertices or surface elements of the model, a location-specific position-uncertainty representation;
(iii) provide, via the display, real-time corrective guidance determined by a spatial distribution of the position-uncertainty representation;
(iv) compute one or more scan-quality metrics including at least one uncertainty-based metric; and
(v) prevent export of the surface model or any measurement derived therefrom until predefined validation thresholds, including a threshold on the uncertainty-based metric, are satisfied, and enable export only when all validation thresholds are satisfied.

17. The system of claim 16, wherein the three-dimensional sensing capability is provided by a visual-inertial simultaneous localization and mapping process without a hardware depth sensor, and wherein the instructions further configure the mobile device to stabilize a metric scale of the model using anthropometric priors.

18. The system of claim 16, wherein the instructions further configure the mobile device to compute a volumetric measurement from the surface model and propagate the position-uncertainty representation through a volume computation function to generate a standard uncertainty of the volume.

19. The system of claim 18, wherein the instructions further configure the mobile device to package the volumetric measurement and the standard uncertainty into a digitally signed, validated data artifact formatted for interoperable exchange with an Electronic Health Record system.

20. A non-transitory computer-readable medium storing instructions that, when executed by one or more processors of a mobile device, cause the mobile device to perform a method for generating a validated three-dimensional model, the method comprising:
(a) capturing a stream of data of an anatomical region;
(b) generating, during the capturing, a three-dimensional surface model from the stream of data and associating, for vertices or surface elements of the model, location-specific position uncertainty values;
(c) computing, during the capturing, one or more scan-quality metrics including at least one metric based on the positional uncertainty values;
(d) providing real-time corrective guidance to a user based on a spatial distribution of the positional uncertainty and values;
(e) preventing export of the three-dimensional surface model or any measurement derived therefrom when at least one of the one or more scan-quality metrics fails to satisfy a predefined validation threshold, and enabling export only when all predefined validation thresholds are satisfied.
